(19) United States
(12) Patent Application Publication
(10) Pub. No.: US 2017/0173036 A1
(43) Pub. Date: Jun. 22, 2017

(54) PHENOTHIAZINE/PHENOTHIAZONE -GRAPHENE OXIDE COMPOSITE

(71) Applicants: B.G. NEGEV TECHNOLOGIES AND APPLICATIONS LTD. (BEN GURION UNIVERSITY), Beer Sheva (IL); YISSUM RESEARCH DEVELOPMENT COMPANY OF THE HEBREW UNIVERSITY OF JERUSALEM LTD., Jerusalem (IL)

(72) Inventors: Lital ALFONTA, Omer (IL); Yehonatan David RAVENNA, Nir am (IL); Ovadia LEV, Jerusalem (IL); Jenny GUN, Jerusalem (IL)

(21) Appl. No.: 15/327,465

(22) PCT Filed: Jul. 21, 2015
(86) PCT No.: PCT/IL2015/050749

§ 371 Date: Jan. 19, 2017

Related U.S. Application Data
(60) Provisional application No. 62/026,734, filed on Jul. 21, 2014.
(60) Provisional application No. 62/164,805, filed on May 21, 2015.
(60) Provisional application No. 62/169,132, filed on Jun. 1, 2015.

(51) Int. Cl. A61K 31/5415; C12Q 1/54

(52) U.S. Cl. CPC A61K 31/5415; C12Q 1/54; C01B 31/043

(57) ABSTRACT

A composite of graphene oxide and PTZ that may further entrap a redox enzyme. This composite can be used for detecting the substrate of the redox enzyme in a sample or for producing an acid byproduct of the enzymatic reaction. Furthermore, provided herein is a kit which includes a composite of graphene oxide and PTZ entrapping a redox enzyme and means for connecting the entrapping composite to a source of electricity.

### FIELD OF INVENTION

This invention is directed to; inter alia, a phenothiazine including derivatives and analogs and/or a phenothiazine (including reduced phenothiazine) including derivatives and analogs (commonly referred to as PTZ) adsorbed on graphene oxide sheets, which mediates the electron transfer between Glucose oxidase and/or Glucose dehydrogenase and an electrode and thus providing a novel hybrid biosensor and a novel PTZ extended release dosage form (graphene oxide sheets and PTZ with or without an enzyme).

### BACKGROUND OF THE INVENTION

Graphene is electrically, mechanically, and chemically stable, in addition graphene is an excellent conductor capable of moving electrons about 100 times faster than silicon and carrying about 100 times more electric current than copper. Thus, many examples of research on production and application of graphene has been carried out recently.

Because of the high demand for blood glucose monitoring, significant research and development efforts have been devoted to producing reliable glucose sensors for in vitro or in vivo applications. Biosensors are based on the direct coupling of a matrix-bound bioactive substance, which is responsible for the specific recognition of the species of interest and a physico-chemical transducer supplying an electric output signal which is processed by the electronic component. In recent years, much effort was invested in enhancement of this electric output in order to achieve more significant and reliable signal.

Due to poor communication between enzymes and electrodes many new techniques utilize nanomaterials to enhance this electronic signal. The unique properties of nanoscale materials offer excellent prospects for interfacing biological recognition events with electronic signal transduction.

Owing to their unique electrical, thermal, and mechanical properties, graphene and its derivatives, including graphene oxide (GO), have attracted ever increasing attention in recent years as a novel class of 3D carbon-based nanomaterials. The reduction of graphene oxide to its reduced form is of great interest in recent years because it partly restores the remarkable electronic properties of graphene and still benefits from the advantages of oxygen-functional groups which provide unique mechanical/chemical properties. Many methods were suggested for reducing GO, including thermal reduction, electrochemical reduction and enzymatic reduction using microorganisms.

### SUMMARY OF THE INVENTION

In one embodiment, the present invention provides a composition comprising graphene oxide and PTZ. In another embodiment, the present invention further provides a composition comprising graphene oxide, PTZ and a redox enzyme. In another embodiment, the present invention further provides a composition comprising graphene oxide, PTZ and glucose oxidase and/or glucose dehydrogenase.

In another embodiment, the present invention further provides a method for detecting, quantifying or both, a carbohydrate, comprising the steps of: contacting a first composition comprising graphene oxide, PTZ and a carbohydrate oxidase and/or carbohydrate dehydrogenase with a second composition comprising said carbohydrate, wherein the carbohydrate is a substrate of the carbohydrate oxidase and/or carbohydrate dehydrogenase; and subjecting the mixture of the first and second compositions to an electric potential, and measuring a current proportional to glucose concentrations, thereby detecting, quantifying or both, a carbohydrate.

In another embodiment, the present invention further provides a method for producing gluconic acid and/or hydrogen peroxide comprising the steps of: contacting a first composition comprising graphene oxide, PTZ and glucose oxidase and/or glucose dehydrogenase with a second composition comprising glucose; and subjecting the mixture of the first and second compositions to an electric potential; thereby producing gluconic acid.

In another embodiment, the present invention further provides a kit comprising graphene oxide, PTZ and a redox enzyme (such as but not limited to glucose oxidase or glucose dehydrogenase) and means for connecting the composition to a source of electricity.

### BRIEF DESCRIPTION OF THE DRAWINGS

FIG. 1. Is a graph of a cyclic voltammogram of GO/PTZ biocomposite encapsulating GOx (9.6 U/ml) in PB solution pH 7 with 0 mM glucose (a), 10 mM glucose (b), 20 mM glucose (c), 30 mM glucose (d), 40 mM glucose (e) and 50 mM glucose (f). Glassy carbon as working electrode, graphite rod as auxiliary electrode and Ag/AgCl (3M KCl) as reference electrode. Potential range was set to 0.6V to −0.2V. The scan rate is 10 mV/s.

FIG. 2. Is a graph of a cyclic voltammogram of GO/PTZ biocomposite encapsulating GOx (9.6 U/ml) in PB solution pH 7 (solid line) or acetate buffer pH 3 (dotted line) without glucose after 5 hours incubation at RT. with Glassy carbon as working electrode, graphite rod as auxiliary electrode and Ag/AgCl (3M KCl) as reference electrode. Potential range was set to 0.6V to −0.2V. The scan rate is 10 mV/s.

FIG. 3. Is a graph of a cyclic voltammogram of PTZ solution with GOx (9.6 U/ml) in PB solution pH 7 with 0 mM glucose (a), 10 mM glucose (b), 20 mM glucose (c), 30 mM glucose (d), 40 mM glucose (e) and 50 mM glucose (f). Glassy carbon as working electrode, graphite rod as auxiliary electrode and Ag/AgCl (3M KCl) as reference electrode. Potential range was set to 0.6V to −0.2V. The scan rate is 10 mV/s.

FIG. 4. Is a graph of a cyclic voltammogram of GO/PTZ biocomposite without the enzyme and glucose as a control (solid line) or GO/PTZ biocomposite encapsulating yeast expressing GOx on their surface without glucose (dotted line) and with 50 mM glucose (dashed line) in PB solution pH 7. Glassy carbon as working electrode, graphite rod as auxiliary electrode and Ag/AgCl (3M KCl) as reference electrode. Potential range was set to 0.6V to −0.2V. The scan rate is 10 mV/s.

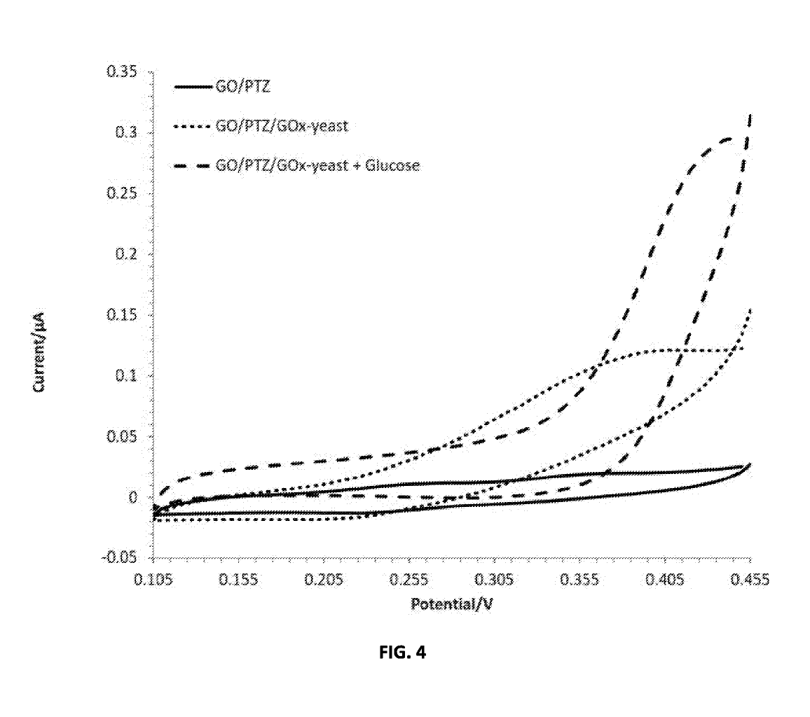

FIG. 5. Is a micrograph showing a SEM image of GO film encapsulating engineered yeast.

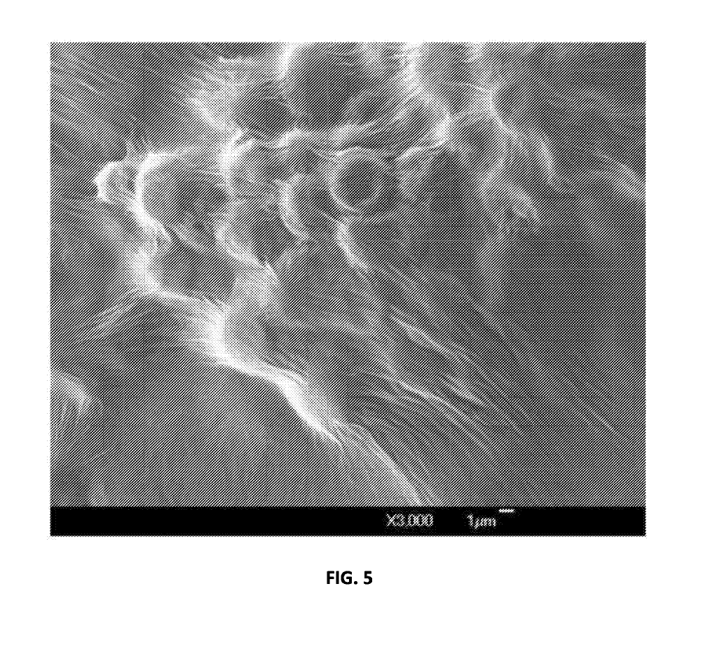

FIG. 6. Is a calibration curve constructed from anodic peak current measurements upon addition of different glucose concentrations to the constructed device, when measuring the peak current linearity of signal appears in the upper range of measured concentrations.

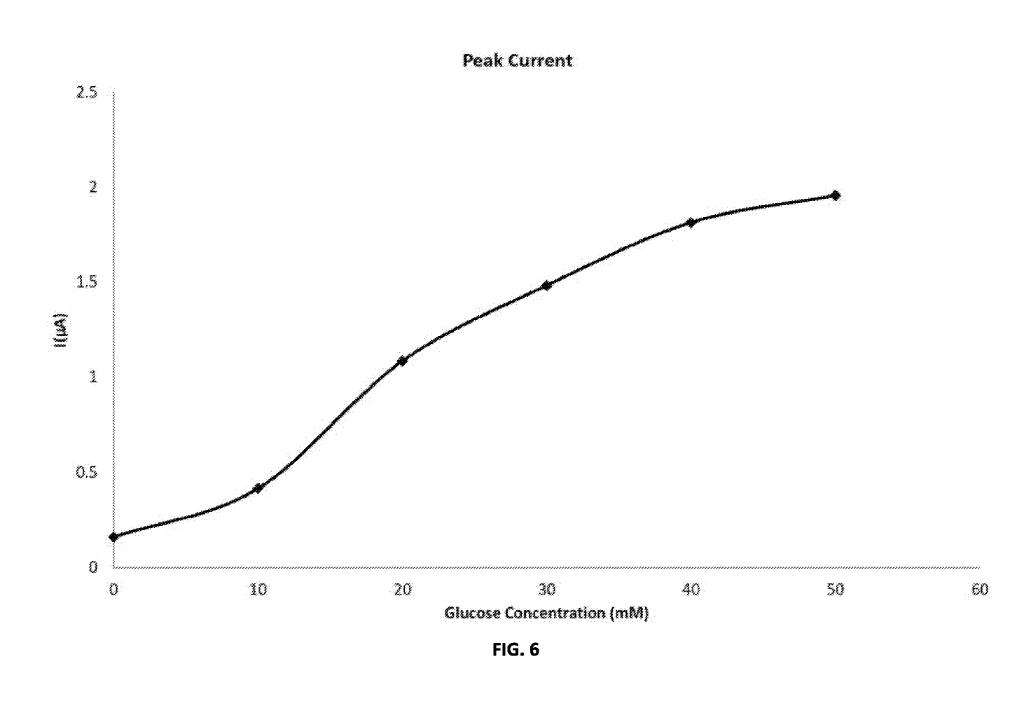

FIG. 7. Is a calibration curve constructed from the potentials of the anodic peak current that also shift with glucose concentration, this is a new concept for glucose sensing. The linearity of the signal appears in the lower range of glucose concentrations. Combinations of the two signals: the shift in potential with the peak current may lead to a better and a more accurate glucose sensing device.

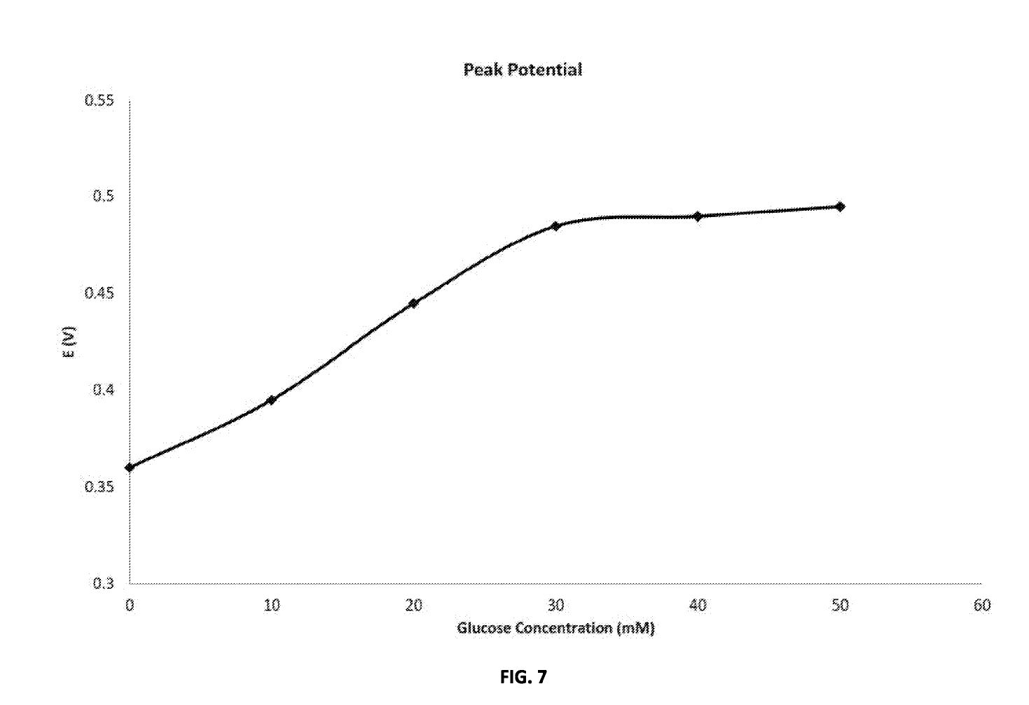

FIG. 8. Are graphs showing cyclic voltammograms (CVs) of GC electrodes: A) (a) rGO/PTZ after oxidation (b) PTZ in solution; B) CV of PTZ extracted from rGO; C) CVs at different scan rates. Inset: scan rate dependent peak potentials of rGO/PTZ/GCE; D) CVs of rGO/PTZ/GCE pH dependence; inset: peak currents vs. pH.

FIG. 9. Is a ESI-high resolution Mass Spectrum of purified phenothiazone in a positive ionization mode.

FIG. 10. Is a ¹³C-NMR spectrum of purified phenothiazine.

FIG. 11. Is a H-NMR spectrum of purified phenothiazine.

FIG. 12. Chemical structure of 1) phenothiazine; 2) 3H-phenothiazine-3-one; 3) a dimer of (1) and (2).

FIG. 13. Are plots showing scan rate dependence of (A) anodic peak potential and (B) anodic peak current for the calculation of the electron transfer rate (α) and average surface concentration (Γ).

FIG. 14. Are plots showing characteristic features of the Raman D and G bands for the sample of GO and for the rGO-PTZ modified sample. (B) XPS of C1s of the same two samples.

FIG. 15. Are chronoamperometric plots showing the response of electrochemically reduced (ER)-rGO/PTZ/FAD-GDH (A) modified glassy carbon electrode; (C) calibration curve for the steady state current upon different glucose concentrations; or cyclic voltammograms of electrochemically reduced (ER)-rGO/PTZ/FAD-GDH modified glassy carbon electrode in the absence of glucose (B, solid line) and in the presence of elevated glucose concentrations (B, dashed lines); (D) calibration curve for the peak oxidation current upon different glucose concentrations.

FIG. 16. Are plots showing the polarization curves (A) and power outputs (B) of (a) ERrGO/PTZ/FAD-GDH; (b) ERrGO/PTZ/GOx; (c) ERrGO/PTZ; modified electrodes, (C) magnified curves (b) and (c) on a lower scale.

FIG. 17. Is a plot showing amperometric response of rGO/PTZ/FAD-GDH/GCE to 3 mM glucose after 24 hours; 8 days; and 14 days.

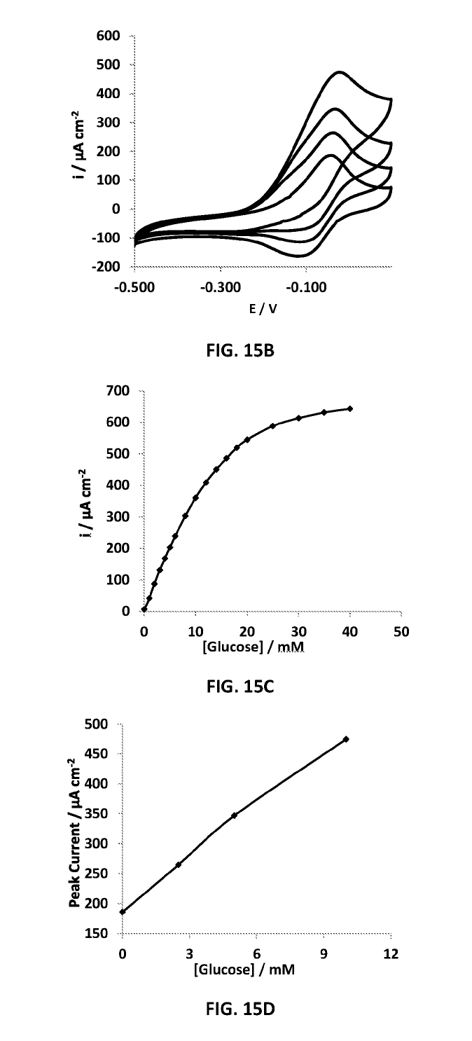

FIG. 18. Is a plot showing chronoamperometric glucose detection using rGO/PTZ/FAD-GDH/GCE for standard additions of 0.5 mM glucose in oxygen saturated and oxygen depleted solution.

FIG. 19. Is a plot showing a selectivity test done by detection of 3.6 mM glucose by rGO/PTZ/FAD-GDH/GCE electrode and afterwards injection of two doses of the interference molecule according to the physiological relevance: 1.67 and 3.3 mM of galactose, 0.3 and 0.6 mM of lactose, 2.9 and 5.8 mM of maltose, 1.67 and 3.3 mM of xylose.

FIG. 20. Is a point-line curve with linear trend-line at the significant release area between 6 to 21 hours and the corresponding trend-line function.

FIG. 21. Is a point curve with an optimal correlated trend-line: the saturation curve.

### DETAILED DESCRIPTION OF THE INVENTION

The term “connected to” that is used to designate a connection of one element to another element includes both a case that an element is “covalently connected to” another element and a case that an element is “electrochemically connected to” another element via another element.

The term “on” that is used to designate a position of one element with respect to another element includes both a case that the one element is adjacent to another element and a case that any other element exists between these two elements.

In one embodiment, provided herein a composition utilized as an electrochemical sensors. In one embodiment, a sensor is coupled to an enzyme reaction. In another embodiment, the composition comprises flavin adenine dinucleotide (FAD) coenzyme molecule. In another embodiment, FAD is bound to the enzyme as described herein. In another embodiment, the term “FAD” includes the reduced FADH₂, the oxidized FAD and a mixture thereof.

In one embodiment, PTZ is a phenothiazine including derivatives and analogs thereof. In one embodiment, derivatives and analogs of PTZ include but are not limited to: methylene blue, phenazine, thionine, azure B²¹, toluidine blueO²², chlorpromazine, prochlorperazine. In one embodiment, PTZ is an oxidized PTZ, such as phenothiazone, PTZ-O, 3H-phenothiazine-3-one, including derivatives and analogs thereof. In one embodiment, PTZ is any phenothiazone and/or derivative as described in European patent No. EP 0115394 B1 which is hereby incorporated by reference in its entirety. In one embodiment, PTZ is a phenothiazine comprises a three-ring structure in which two benzene rings are linked by nitrogen and sulfur. In one embodiment, PTZ is chlorpromazine. In one embodiment, PTZ is prochlorperazine. In one embodiment, PTZ is any derivative of phenothiazine or phenothiazone known to one of skill in the art. In one embodiment, PTZ is a mixture of phenothiazine and phenothiazone. In one embodiment, PTZ is: a phenothiazine, a phenothiazine analogue, phenothiazine derivative, a phenothiazone, a phenothiazone analogue, phenothiazone derivative, or any combination thereof.

In one embodiment, PTZ is enriched for phenothiazone, and phenothiazine is in trace amount. In one embodiment, PTZ comprises at least 95% phenothiazone. In one embodiment, PTZ comprises at least 98% phenothiazone. In one embodiment, PTZ comprises at least 98% phenothiazone. In one embodiment, PTZ comprises less than 5% phenothiazine. In one embodiment, PTZ comprises less than 2% phenothiazine. In one embodiment, PTZ comprises less than 1% phenothiazine. In one embodiment, PTZ comprises less than 0.5% phenothiazine. In one embodiment, PTZ comprises less than 0.05% phenothiazine. In one embodiment, PTZ is at least 90% in a reduced form. In one embodiment, PTZ is at least 95% in a reduced form. In one embodiment, PTZ is at least 98% in a reduced form. In one embodiment, PTZ is at least 99% in a reduced form. In one embodiment, reacting PTZ and GO results in oxidized PTZ and at least partially reduced GO. In one embodiment, reacting PTZ and reduced graphene oxide results in no or little oxidation-reduction reaction.

The term “comprises or includes” and/or “comprising or including” used in the document means that one or more other components, steps, operation and/or existence or addition of elements are not excluded in addition to the described components, steps, operation and/or elements unless context dictates otherwise.

In one embodiment, GO is a reduced form of GO as further described herein. In one embodiment, the term “GO” includes a reduced form of GO or rGO. In one embodiment, GO is at least partially reduced GO. In one embodiment, the composition of the invention includes phenothiazine, GO and optionally an enzyme as described herein. In one embodiment, the composition of the invention includes phenothiazone, rGO and optionally an enzyme as described herein. In one embodiment, the composition of the invention includes: phenothiazine, phenothiazone, GO, rGO or any combination thereof. In one embodiment, the composition of the invention includes: phenothiazine, a phenothiazine analogue, phenothiazine derivative, phenothiazone, a phenothiazone analogue, phenothiazone derivative, GO, rGO or any combination thereof.

The term “about or approximately” or “substantially” are intended to have meanings close to numerical values or ranges specified with an allowable error and intended to prevent accurate or absolute numerical values disclosed for understanding of the present disclosure from being illegally or unfairly used by any unconscionable third party.

The term “combination of” included in Markush type description means mixture or combination of one or more components, steps, operations and/or elements selected from the group consisting of components, steps, operation and/or elements described in Markush type and thereby means that the disclosure includes one or more components, steps, operations and/or elements selected from the Markush group.

The term “graphene” is a polycyclic aromatic molecule formed by covalently bonding multiple carbon atoms. The covalently bonded carbon atoms form a six-member carbon ring as a repeating unit and may further includes a five-member carbon ring and/or a seven-member carbon ring. Therefore, a sheet made of the graphene can be seen as, but not limited to, a monolayer of covalently bonded carbon atoms. The sheet made of the graphene may have various structures depending on a content of the five-member carbon ring and/or the seven-member carbon ring which may be included in the graphene. If a sheet made of the graphene is configured as a monolayer, multiple sheets may be stacked to form multiple layers. A side end of the graphene sheet may be saturated with, but not limited to, a hydrogen atom. Graphene oxide, according to some embodiments, is reduced graphene oxide.

In one embodiment, provided herein a composite of graphene oxide and PTZ for mediating the electron transfer between carbohydrate redox enzyme (such as glucose oxidase and/or glucose dehydrogenase and optionally a cofactor such as FAD when necessary) and an electrode. In one embodiment, provided herein a hybrid biosensor comprising graphene oxide and PTZ adapted to transfer an electron between carbohydrate redox enzyme (such as glucose oxidase and/or glucose dehydrogenase and optionally a cofactor such as FAD when necessary) and an electrode. In one embodiment, provided herein a composition comprising graphene oxide, PTZ, a carbohydrate and an electrode. In one embodiment, provided herein a kit comprising: (a) composition comprising graphene oxide, PTZ and a carbohydrate redox enzyme; and (b) an electrode. In one embodiment, provided herein a kit comprising: (a) composition comprising graphene oxide, PTZ and a carbohydrate redox enzyme immobilized on an electrode; and (b) means for connecting the electrode to an electric power source (such as an electrical wire or cable). In one embodiment, provided herein a composition comprising graphene oxide, PTZ and carbohydrate redox enzyme coupled to an electrode. In another embodiment, a redox enzyme and PTZ in combination with rGO is used herein for sensing/biosensing glucose in a body fluid sample (ex-vivo and/or in-vitro).

In one embodiment, carbohydrate sensing is sensing glucose in a body fluid such as the blood. In one embodiment, a body fluid to be sensed/assayed comprises at least 1 mg/dl. In one embodiment, a body fluid to be sensed/assayed comprises at least 5 mg/dl. In one embodiment, a body fluid to be sensed/assayed comprises at least 15 mg/dl. In one embodiment, a body fluid to be sensed/assayed comprises at least 25 mg/dl. In one embodiment, a body fluid to be sensed/assayed comprises at least 50 mg/dl. In one embodiment, a body fluid to be sensed/assayed comprises up to 5000 mg/dl. In one embodiment, a body fluid to be sensed/assayed comprises up to 1000 mg/dl. In one embodiment, a body fluid to be sensed/assayed comprises up to 1000 mg/dl. In one embodiment, a body fluid to be sensed/assayed comprises up to 700 mg/dl. In one embodiment, a body fluid to be sensed/assayed comprises up to 400 mg/dl.

In one embodiment, carbohydrate sensing according to the invention has an accuracy of at least +/−25 mg/dl in 95% of the results. In one embodiment, carbohydrate sensing according to the invention has an accuracy of at least +/−20 mg/dl in 95% of the results. In one embodiment, carbohydrate sensing according to the invention has an accuracy of at least +/−15 mg/dl in 95% of the results. In one embodiment, carbohydrate sensing according to the invention has an accuracy of at least +/−10 mg/dl in 95% of the results. In one embodiment, carbohydrate sensing according to the invention has an accuracy of at least +/−5 mg/dl in 95% of the results. In one embodiment, carbohydrate sensing according to the invention has an accuracy of at least +/−2 mg/dl in 95% of the results. In one embodiment, carbohydrate sensing according to the invention has an accuracy of at least +/−15% in 95% of the results. In one embodiment, carbohydrate sensing according to the invention has an accuracy of at least +/−12% in 95% of the results. In one embodiment, carbohydrate sensing according to the invention has an accuracy of at least +/−10% in 95% of the results. In one embodiment, carbohydrate sensing according to the invention has an accuracy of at least +/−5% in 95% of the results.

In another embodiment, PTZ comprises less than 10% reduced form of PTZ. In another embodiment, PTZ comprises less than 8% reduced form of PTZ. In another embodiment, PTZ comprises less than 7% reduced form of PTZ. In another embodiment, PTZ comprises less than 5% reduced form of PTZ. In another embodiment, PTZ comprises less than 3% reduced form of PTZ. In another embodiment, PTZ comprises less than 1% reduced form of PTZ. In another embodiment, PTZ comprises less than 0.5% reduced form of PTZ. In another embodiment, PTZ comprises less than 0.10% reduced form of PTZ.

In another embodiment, PTZ includes at least 85% oxidized PTZ or PTZ-O. In another embodiment, PTZ includes at least 90% oxidized PTZ or PTZ-O. In another embodiment, PTZ includes at least 94% oxidized PTZ or PTZ-O. In another embodiment, PTZ includes at least 95% oxidized PTZ or PTZ-O. In another embodiment, PTZ includes at least 97% oxidized PTZ or PTZ-O. In another embodiment, PTZ includes at least 99% oxidized PTZ or PTZ-O. In another embodiment, PTZ includes at least 99.8% oxidized PTZ or PTZ-O. In another embodiment, PTZ includes at least 90% oxidized PTZ or PTZ-O. In another embodiment, PTZ includes at least 99.9% oxidized PTZ or PTZ-O.

In one embodiment a composition as described herein further comprises flavin adenine dinucleotide (FAD). In one embodiment, provided herein a composition comprising graphene oxide/rGO and carbohydrate dehydrogenase. In one embodiment, provided herein a composition comprising graphene oxide/rGO and glucose dehydrogenase (GDH). In another embodiment, GDH or the enzyme is flavin adenine dinucleotide (FAD) dependent glucose dehydrogenase (FAD-GDH). In another embodiment, GDH or the enzyme is FAD-GDH (E.C. 1.1.5.9). In some embodiments, glucose dehydrogenase can be derived from fungal source such as aspergillus genus, for example aspergillus oryzae, aspergillus niger, aspergillus terreus, Aspergillus carbonarius, Aspergillus foetidus, Aspergillus ustus, Aspergillus foetidus var. Pallidus, Aspergillus flavus, Aspergillus flavus var. Columnaris, Aspergillus aculeatus, Aspergillus phoenicis, Aspergillus brunneo-uniseriatus and the likes, or from Penicillium genus such as Penicillium hlacinoechinulatum, Penicillium italicum Penicillium rugulosum, Penicillium expansum, Penicillium jensenii, Penicillium clavigerum, Penicillium capsulatum, Penicillium velutinum, Penicillium janczewskii, Penicillium abeanum, Penicillium isarhforme, Penicillium raciborskii, Penicillium echinulatumvar echinulatum, Penicillium lanosoviride, Penicillium palitans, Penicillium resticulosum, Penicillium sohtum, or from any other appropriate fungal source. The glucose dehydrogenase of the present invention can be derived from a bacterial source, for example from Burkholderia sp. for example Burkholderia Cepacia, Burkholderia lata, Burkholderia terrae, or other bacterial source such as Pseudomonas sp. such as Pseudomonas denitrificans, Pseudomonas syringae, Pseudomonas putida Pseudomonas fluorescens and the like, or any other appropriate bacterial source such as Coli spp., Yersinia spp., Herbaspirillum spp, and the like.

In one embodiment, provided herein a composition comprising graphene oxide/rGO and carbohydrate dehydrogenase without a cofactor. In one embodiment, provided herein a composition comprising graphene oxide/rGO and carbohydrate dehydrogenase and/or carbohydrate oxidase. In one embodiment, a composition as described herein further comprises a cofactor such as FAD, NAD, PQQ or any combination thereof. In one embodiment, a composition comprising GDH as an enzyme requires a cofactor or FAD.

In one embodiment, provided herein a composition comprising an electrode coated with rGO/PTZ/GDH (glucose dehydrogenase) or rGO/PTZ/Glucose-Oxidase. In another embodiment, provided herein a composition comprising a carbon electrode coated with rGO/PTZ/GDH or rGO/PTZ/Glucose-Oxidase film. In one embodiment, provided herein a composition comprising a glassy carbon electrode (GCE) coated with rGO/PTZ/GDH or rGO/PTZ/Glucose-Oxidase film used for biosensing as well as for bio-fuel cell applications. In one embodiment, provided herein a composition comprising rGO/PTZ/GDH to be applies to an anode. In one embodiment, provided herein a biofuel cell wherein the anode comprises rGO/PTZ/GDH or rGO/PTZ/Glucose-Oxidase.

In one embodiment, provided herein a composition comprising a film modified electrode comprising of an enzyme, mediator and rGO. In one embodiment, provided herein a composition comprising a film modified electrode comprising of an enzyme and rGO. In one embodiment, provided herein a composition comprising a film modified electrode comprising of rGO and FAD-GDH. In one embodiment, the enzyme, GO or rGO and possibly a co-factor are immobilized on the surface of the electrode.

In one embodiment, provided herein a composite of graphene oxide and PTZ that further entraps and/or coats a redox enzyme. This composite can be used, in some embodiments, for detecting the substrate of the redox enzyme (such as but not limited to glucose) in a sample (such as but not limited to blood or urine) or for producing an acid byproduct of the enzymatic reaction.

In one embodiment, provided herein a controlled release or extended release composition comprising PTZ and GO. In one embodiment, provided herein a controlled release composition comprising PTZ and GO without a redox enzyme. In one embodiment, provided herein a controlled release composition comprising PTZ, GO without a redox enzyme. In one embodiment, provided herein a controlled release composition comprising PTZ, carbohydrate redox enzyme (such as glucose oxidase and/or glucose dehydrogenase) and GO. In one embodiment, provided herein an extended release composition comprising PTZ and GO. In one embodiment, PTZ is released in an extended manner from GO. In one embodiment, PTZ is released from the GO under catalytic reaction and is dependent on glucose concentrations in the presence of the enzyme glucose oxidase or glucose dehydrogenase. In one embodiment, PTZ release from GO is controlled by glucose addition or glucose concentration. In one embodiment, provided herein a controlled release psychiatric dosage form. In another embodiment, provided herein a PTZ drug delivery platform. In one embodiment, provided herein a method for treating a psychiatric disease such as a psychotic disease, comprising administering a composition as described herein.

In one embodiment, provided herein a controlled release or extended release composition comprising PTZ and graphene oxide (GO). In one embodiment, a controlled release or extended release composition is an oral dosage form. In one embodiment, a controlled release or extended release composition is provided in the form of a tablet. In one embodiment, provided herein a method for extending the release of PTZ in a physiological environment, comprising the step of combining said PTZ and GO. In one embodiment, a physiological environment is any live tissue. In one embodiment, a physiological environment comprises a bodily fluid. In one embodiment, a physiological environment comprises a carbohydrate. In one embodiment, a physiological environment comprises glucose.

In one embodiment, provided herein use of a composition of the invention such as a composition comprising PTZ and graphene oxide (GO) for the preparation of a medicament for treating a psychiatric disease. In one embodiment, provided herein a method for treating a psychiatric disease in a subject in need thereof, comprising administering to the subject a composition comprising PTZ and graphene oxide (GO). In one embodiment, a composition as described herein is extremely useful as GO is a biocompatible substance which: (1) converts most PTZ to PTZ-O; and (2) at the same time efficiently extends the release of biologically active PTZ-O.

In one embodiment, a combined composition comprising GO/PTZ-O releases in-vivo or under physiological conditions, at least 70% of PTZ-O within 2 to 30 hours. In one embodiment, a combined composition comprising GO/PTZ-O releases in-vivo or under physiological conditions, at least 70% of PTZ-O within 4 to 24 hours. In one embodiment, a combined composition comprising GO/PTZ-O releases in-vivo or under physiological conditions, at least 70% of PTZ-O within 6 to 20 hours. In one embodiment, a combined composition comprising GO/PTZ-O releases in-vivo or under physiological conditions, at least 75% of PTZ-O within 4 to 24 hours. In one embodiment, a combined composition comprising GO/PTZ-O releases in-vivo or under physiological conditions, at least 80% of PTZ-O within 4 to 24 hours. In one embodiment, a combined composition comprising GO/PTZ-O releases in-vivo or under physiological conditions, at least 85% of PTZ-O within 4 to 24 hours. In one embodiment, a combined composition comprising GO/PTZ-O releases in-vivo or under physiological conditions, at least 90% of PTZ-O within 4 to 24 hours. In one embodiment, a combined composition comprising GO/PTZ-O releases in-vivo or under physiological conditions, at least 80% of PTZ-O within 20 hours. In one embodiment, a combined composition comprising GO/PTZ-O releases in-vivo or under physiological conditions, at least 80% of PTZ-O within 24 hours. In one embodiment, a combined composition comprising GO/PTZ-O releases in-vivo or under physiological conditions, at least 90% of PTZ-O within 22 hours. In one embodiment, a combined composition comprising GO/PTZ-O releases in-vivo or under physiological conditions, at least 90% of PTZ-O within 20 hours. In one embodiment, the term “releases” is release free water-soluble PTZ-O. In one embodiment, the term “releases” is release biologically active PTZ-O. In one embodiment, physiological conditions include any composition comprising cells, derived from cells, tissue, derived from a tissue or a bodily fluid. In one embodiment, physiological conditions include the circulatory system, the gastro-intestinal system, or any other living tissue or organ.

In one embodiment, provided herein a method for preparing a composition comprising oxidized-PTZ and rGO comprising the step of mixing oxidized-PTZ with rGO. In one embodiment, a method for preparing a composition comprising oxidized-PTZ and rGO further comprises the step of mixing GO aqueous solution with PTZ dissolved in an organic solvent. In one embodiment, a method for preparing a composition comprising oxidized-PTZ and rGO further comprises dialyzing the mix in water for a predetermined time, in order to remove the organic solvent from the solution and/or non-adsorbed PTZ or oxidized PTZ. In one embodiment, a method for preparing a composition comprising oxidized-PTZ and rGO further comprises reducing the composition electrochemically by operating an appropriate voltage on an electrode in the solution for a predetermined time. In one embodiment, remainders of the un-oxidized form of PTZ remain in the system in trace amounts, as well as other oxidized species such as a dimer of phenothiazine-phenothiazone, yet the dominant species is the oxidized-PTZ (phenothiazone).

In one embodiment, provided herein a method for preparing a strip-like biosensor comprising the steps of: mixing GO aqueous solution with PTZ dissolved in an organic solvent, incubating the mix for 30 seconds, 50 minutes, 2 hours, 5 hours, to overnight, dialyzing the mix in water for 30 seconds to a month. minutes (in order to remove the organic solvent and non-adsorbed PTZ), adding a redox enzyme to reach a desired concentration, applying a thin layer of the resulting composition to an electrode and optionally electrochemically reducing the composition by applying an appropriate voltage to the electrode. In one embodiment, dialyzing the mix in water is for 1 week to 3 weeks. In one embodiment, dialyzing the mix in water is for 12 to 16 days.

In one embodiment, a method for preparing a strip-like biosensor or a method for preparing a composition comprising oxidized-PTZ and rGO is a method for preparing an anode of a fuel-cell. In one embodiment, a method for preparing a strip-like biosensor or a method for preparing a composition comprising oxidized-PTZ and rGO is a method for producing oxidized-PTZ. In one embodiment, a method for preparing a strip-like biosensor or a method for preparing a composition comprising oxidized-PTZ and rGO is a method for enriching oxidized-PTZ within PTZ (a mixture of both oxidized and reduced PTZ). In one embodiment, a method for preparing a strip-like biosensor or a method for preparing a composition comprising oxidized-PTZ and rGO is a method for and extending the release of PTZ as a drug.

In another embodiment, a composition as described herein is mixed with a bodily fluid. In another embodiment, a composition as described herein further comprises a bodily fluid such as but not limited to blood. In another embodiment, a composition as described herein comprises a sample comprising body fluid and rGO/PTZ/GDH or rGO/PTZ/Glucose-Oxidase. In another embodiment, a composition as described herein comprises a sample comprising body fluid and rGO/PTZ/Glucose-Oxidase In another embodiment, a composition as described herein comprises a body fluid sample and rGO/PTZ/GDH or rGO/PTZ/Glucose-Oxidase modified glassy carbon electrode. In another embodiment, a composition as described herein comprises glucose, a body fluid sample and rGO/PTZ/GDH or rGO/PTZ/Glucose-Oxidase. In another embodiment, a composition as described herein comprises glucose and rGO/PTZ/GDH or rGO/PTZ/Glucose-Oxidase. In another embodiment, GDH is of a fungal source or a bacterial source.

In one embodiment, an extended or controlled release composition as described herein is utilized in treating a subject afflicted with a psychotic condition. In one embodiment, an extended or controlled release composition as described herein is utilized in treating a subject in need of an antihistaminic therapy. In one embodiment, an extended or controlled release composition as described herein is utilized in treating a subject in need of an inhibitor of leukotriene biosynthesis therapy. In one embodiment, an extended or controlled release composition as described herein is utilized in treating a subject afflicted with pain. In one embodiment, an extended or controlled release composition as described herein is utilized in treating a subject afflicted with a skin condition. In one embodiment, an extended or controlled release composition as described herein is utilized in treating a subject afflicted with an inflammatory condition such as but not limited to a skin inflammatory condition. In one embodiment, an extended or controlled release composition as described herein is utilized in treating a subject afflicted with allergy. In one embodiment, an extended or controlled release composition as described herein is utilized in treating a subject afflicted with a pulmonary disease. In one embodiment, an extended or controlled release composition as described herein is utilized in treating a subject afflicted with asthma. In one embodiment, an extended or controlled release composition as described herein is utilized in treating a subject afflicted with a cardiovascular disorder. In one embodiment, an extended or controlled release composition as described herein is utilized in treating a subject afflicted with inflammation.

In one embodiment, provided herein a composition comprising graphene oxide and PTZ. In another embodiment, provided herein a composite comprising graphene oxide and PTZ. In one embodiment, provided herein a composition comprising graphene oxide, PTZ and a redox enzyme.

In one embodiment, provided herein a composition comprising graphene oxide, PTZ and a carbohydrate oxidase. In one embodiment, the carbohydrate oxidase is glucose oxidase. In one embodiment, glucose oxidase is a yeast glucose oxidase. In one embodiment, glucose oxidase is plant glucose oxidase. In one embodiment, glucose oxidase is a bacterial glucose oxidase. Glucose oxidase, in some embodiments, is a fungi enzyme. In some embodiments, glucose oxidase is expressed on the surface of yeast. In another embodiment glucose oxidase is a pure fungal enzyme.

In one embodiment, a composite of graphene oxide (GO) and PTZ entraps and/or surrounds a carbohydrate oxidase. In one embodiment, a composite of graphene oxide (GO) and PTZ entraps and/or surrounds glucose oxidase. In one embodiment, a composite and/or composition as described herein is a bioreactor (bio-fuel cell) and/or biosensor. In one embodiment, the redox enzyme is provided within a cell. In one embodiment, a composite of GO and PTZ entraps and/or surrounds a cell comprising a carbohydrate oxidase and/or reductase. In one embodiment, a cell is a eukaryotic cell. In one embodiment, a cell is a prokaryotic cell. In one embodiment, a cell is a yeast cell. In one embodiment, a cell is a bacterial cell.

In one embodiment, phenothiazine and/or reduced phenothiazine incubated with GO is converted to phenothiazone (oxidized) and/or reduced phenothiazine. In one embodiment, phenothiazine and/or reduced phenothiazine incubated with graphene oxide is converted to phenothiazone and/or reduced phenothiazine while GO is converted to a reduced form-rGO. In one embodiment, phenothiazine and/or reduced phenothiazine incubated with GO was oxidized to phenothiazone (PTZ-O) and in return GO was reduced to rGO. In one embodiment, the identity of the oxidized phenothiazine was determined by extracting the PTZ from GO with methanol followed by filter paper separation, after which the sample was injected into LC-MS for separation and analysis—NMR and cyclic voltammetry has confirmed that the extracted molecule is phenothiazone (PTZ-O). In one embodiment, contacting GO and PTZ results in a mixture having a ratio (weight/weight or weight percent) of phenothiazone to phenothiazine of at least 9:1. In one embodiment, contacting GO and phenothiazine results in a mixture or a composition having a ratio (weight/weight or weight percent) of phenothiazone to phenothiazine of at least 9:1. In one embodiment, contacting GO and phenothiazine results in a mixture or a composition having a ratio (weight/weight or weight percent) of phenothiazone to phenothiazine of at least 10:1. In one embodiment, contacting GO and phenothiazine results in a mixture or a composition having a ratio (weight/weight or weight percent) of phenothiazone to phenothiazine of at least 15:1. In one embodiment, contacting GO and phenothiazine results in a mixture or a composition having a ratio (weight/weight or weight percent) of phenothiazone to phenothiazine of at least 20:1. In one embodiment, contacting GO and phenothiazine results in a mixture or a composition having a ratio (weight/weight or weight percent) of phenothiazone to phenothiazine of at least 50:1. In one embodiment, contacting GO and phenothiazine results in a mixture or a composition having a ratio (weight/weight or weight percent) of phenothiazone to phenothiazine of at least 100:1.

In another embodiment, reduced PTZ is oxidized to oxidized PTZ (PTZ-O) by GO, which in turn is at least partially reduced or reduced to rGO. In another embodiment, a composition and/or composite as described herein comprises a combination of reduced PTZ and oxidized PTZ. In another embodiment, a composition and/or composite as described herein comprises a dimer of a combination of reduced PTZ and PTZ-O. In another embodiment, the dimer in a composition and/or composite as described herein is present in smaller quantities in the GO matrix and the dominant species is PTZ-O.

In one embodiment, a composition as described herein further comprises a substrate to be detected by the redox enzyme. In one embodiment, a composition as described herein further comprises the substrate of the redox enzyme. In one embodiment, a composition as described herein further comprises glucose. In one embodiment, a composition as described herein further comprises the byproducts of an enzymatic reaction of the carbohydrate and the carbohydrate oxidase and/or the carbohydrate dehydrogenase. In one embodiment, a composition as described herein further comprises the byproducts of an enzymatic reaction of the glucose oxidase and/or glucose dehydrogenase. In one embodiment, a composition as described herein further comprises gluconic acid.

In one embodiment, provided herein a method for detecting, quantifying or both, a carbohydrate in a sample, comprising the steps of: (a) contacting a composition comprising graphene oxide, PTZ and a carbohydrate redox enzyme with a sample; and (b) subjecting the mixture of the composition and the sample to an electric potential, wherein the carbohydrate is a substrate of the carbohydrate redox enzyme; thereby detecting, quantifying or both, a carbohydrate in a sample. In one embodiment, provided herein a method for detecting glucose in blood and/or urine.

In one embodiment, provided herein a method for producing gluconic acid and hydrogen peroxide in the presence of oxygen comprising the steps of: (a) contacting a first composition comprising graphene oxide, PTZ and a glucose oxidase with a second composition comprising glucose; and (b) subjecting the mixture of the first and second compositions to an electric potential, thereby producing gluconic acid and hydrogen peroxide. In one embodiment, the second composition is comprises a bodily fluid.

In one embodiment, provided herein a method for producing an acid of a carbohydrate comprising the steps of: (a) contacting a first composition comprising graphene oxide, PTZ and a carbohydrate oxidase with a second composition comprising a carbohydrate, wherein the carbohydrate is a substrate of the carbohydrate oxidase; and (b) subjecting the mixture of the first and second compositions to an electric potential, thereby producing gluconic acid and hydrogen peroxide.

In one embodiment, provided herein a kit comprising a composition as described herein and means for connecting the composition to a source of electricity. In one embodiment, provided herein a kit comprising a composition as described herein, means for connecting the composition to a source of electricity, and an instructions manual. In one embodiment, provided herein a kit comprising a composition as described herein applied on or connected to an electrode.

A composition, a biosensor, a composite or a biocomposite includes, in some embodiments, a source electrode and a drain electrode. A composition, a biosensor, a composite or a biocomposite includes, in some embodiments, a source electrode and a drain electrode existing on the same plane and including the composition as described herein.

In another embodiment, the invention provides a GO/PTZ biocomposite for glucose biosensing. In one embodiment, the present invention provides a PTZ adsorbed on GO sheets for mediating electron transfer between a redox enzyme such as carbohydrate oxidase and an electrode and thus achieving a novel hybrid biosensor. In another embodiment, the invention provides a composition comprising GO, Glucose Oxidase enzyme, and PTZ. In another embodiment, the invention provides a composition wherein GO adsorbed PTZ by π-π interactions and encapsulates a carbohydrate oxidase enzyme, such as but not limited to glucose oxidase. In another embodiment, the invention provides GO which adsorbs large quantities of PTZ. In another embodiment, a composition as described herein is a biocomposite and/or biosensor. In another embodiment, a composition as described herein mediates electron transfer between redox enzymes and electrodes. In another embodiment, a composition as described herein interacts with redox enzymes and microorganisms.

In another embodiment, Graphene oxide (GO) may be modified to reduced Graphene oxide (rGO). Graphene oxide is produced, in some embodiments, by conventional methods which include subjecting graphene to oxidizing reagents, thus introducing oxygen containing functional groups such as carboxylic acids, ketones and aldehydes into graphene. In another embodiment, GO may be reduced bioelectrochemically. In another embodiment, rGO is stabilized by cathodic potential cycling.

In another embodiment, a composition as described herein is the form of a composite entrapping or in contact with a carbohydrate oxidase or carbohydrate dehydrogenase. In another embodiment, a composition as described herein is in the form of an electrode. In another embodiment, a composition as described herein is coupled and/or immobilized on or to an electrode. In another embodiment, a composition as described herein is in the form or coupled to an electrode such as glassy carbon electrode (GCE).

In another embodiment, a composition as described herein is a fabricated glucose biosensor. In another embodiment, a composition as described herein is a fabricated glucose biosensor having a glucose detection limit of 0.005 mM. In another embodiment, a composition as described herein is a fabricated glucose biosensor having a glucose detection limit of 0.01 mM. In another embodiment, a composition as described herein is a fabricated glucose biosensor having a glucose detection limit of 0.02 mM. In another embodiment, a composition as described herein is a fabricated glucose biosensor having a glucose detection limit of 0.5 mM. In another embodiment, a composition as described herein is a fabricated glucose biosensor having a glucose detection limit of 1 mM.

In one embodiment, a composition of the present invention is a biosensor wherein the composition is formed on a substrate. In one embodiment, a composition of the present invention is a biosensor wherein the composition is formed on an electrode. In one embodiment, a composition of the present invention is a biosensor applied on a surface adapted to connect to a source of electricity. In some embodiments, “biosensor” is a sensor for detecting presence of a bio material such as a carbohydrate or a carbohydrate acid. In one embodiment, the composition of the invention is connected to a device that apply potential (such as potentiostat). The biosensor identifies a kind of a material by bonding a target material and a probe material. The target material is a target object to be sensed, and the probe material is a material capable of being specifically and selectively bonded to the target material. The biosensor may have, in some embodiments, various detection methods. In one embodiment, the present invention provides an electrochemical biosensor. In one embodiment, a biosensor electrochemically senses a carbohydrate such as but not limited to glucose.

In another embodiment, a composition and a system comprising the composition and an electric system coupled to a voltage (via an electric wire or cable) and/or current detectors/meters are used for glucose monitoring. In another embodiment, a composition and a system comprising the composition and an electric system coupled to a voltage and/or current detectors/meters are used for blood glucose monitoring. In another embodiment, a biosensor or glucose detector of the invention is composed of the composition as described herein and a physico-chemical transducer supplying an electric output signal which is processed by an electronic component.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes Cellis, J. E., ed. (1994); “Culture of Animal Cells—A Manual of Basic Technique” by Freshney, Wiley-Liss, N. Y. (1994), Third Edition; “Current Protocols in Immunology” Volumes Coligan J. E., ed. (1994); Stites et al. (eds), “Basic and Clinical Immunology” (8th Edition), Appleton & Lange, Norwalk, Conn. (1994); Mishell and Shiigi (eds), “Strategies for Protein Purification and Characterization—A Laboratory Course Manual” CSHL Press (1996); all of which are incorporated by reference. Other general references are provided throughout this document.

### Materials and Methods

### Bioelectrocatalysis

GO solution was diluted with phosphate buffer (pH 7; PB) (1:1) for the control or different concentrations of glucose for the biocatalysis. 10 μM of free commercial glucose oxidaze (5 mg/ml; GOx) was added to the solution to achieve final enzyme concentration of 0.05 mg/ml (9.6 units/ml). All samples, in the volume of 1 ml, were incubated at room temperature (R.T.) for at least 1 h to ensure well diffusion of all components. A conventional three electrode set-up was assembled with graphite stick as counter electrode, glassy carbon disk electrode (3 mm in diameter; ALS, Tokyo, Japan) as working electrode and Ag/AgCl (NaCl 3M) electrode as reference electrode (ALS, Tokyo, Japan).

Cyclic voltammetry (CV) was performed with PalmSense potentiostat (Palm Instruments, Houten, The Netherlands) between −0.2 V to 0.6 V at scan rate of 10 mV/sec. Prior to cell conduction, the GCE was polished with alumina slurry (0.05 μm), washed with distilled water (DW), sonicated in DW for 5 min and washed again with DW. The counter electrode was polished with sand paper and washed with ethanol and DW. The three electrodes were immersed directly in the sample solution and the measurements were conducted under aerobic conditions. Between every sample measurement, the GCE was polished with alumina slurry to avoid PTZ residues adsorbed on the electrode, washed with DW and dried with air. The counter and reference electrodes were washed with DW.

### Graphene Oxide (GO) Synthesis

GO was synthesized from exfoliated graphite by a modified Hummers method. First, exfoliated graphite powder (1 g) was added to a solution of K₂S₂O₈ (1.67 g) and P₂O₅ (1.67 g) in 8 mL concentrated H₂SO₄. The mixture was kept at 80° C. for 4.5 h on a hot plate. After the mixture was cooled to room temperature, it was diluted with 0.35 L of deionized water (DIW) and filtered. Then the preoxidized material was washed with DIW and dried at 60-70° C. overnight. Next, preoxidized carbon was redispersed in 40 mL of concentrated H₂SO₄ with the mixture kept in an ice bath. Subsequently, 5 g of potassium permanganate were added gradually under constant stirring to avoid overheating. The mixture was stirred at 35° C. for 2 h and then slowly diluted with 80 mL of DIW upon cooling in the ice bath.

The mixture was stirred for an additional 2 h and then 250 mL more DIW were added, followed by the addition of 6 mL of 30% H₂O₂ to react with the excess of permanganate. The color of the solution changed to yellow after addition of the peroxide. The oxidized product was filtered and washed with 100 mL HCl (1:10) to remove metal ion impurities, followed by washing with 300 mL of DIW and by dialysis to remove the acid. A dispersion of GO in water was prepared by dispersing the oxidized material in DIW in an ultrasound bath for 2 h. Aqueous GO dispersions were stable for at least a few months. The concentration of GO in this aqueous solution was 0.99 g per 100 ml of the solution.

### PTZ Modified GO Synthesis

35 mL of 1% GO, was centrifuged till the separation of GO colloid (app 15 mL) and transparent water. Water was decanted and to the remaining GO colloid 20 mL of MeCN solution with 100 mg of PTZ dissolved in it was added.

The mixture was left overnight in room temperature after that centrifuged and the solid fraction was transferred into dialysis membrane. The washing of the mixture in the dialysis membrane was conducted for 2 weeks in water with twice a day change of the water.

### Electrode Fabrication

Prior to electrode modification, glassy carbon electrode (GCE) (3 mm in diameter; ALS, Tokyo, Japan) was polished with 0.05 μm alumina slurry, rinsed with DW, sonicated in DW for 5 minutes and dried with pure nitrogen. PTZ modified graphene oxide hydrogel was mixed with 10 mg/mL FAD-GDH in PB pH 7 to generate a final enzyme loading of 5 mg/mL. For control experiments the same concentration of glucose oxidase or 0.1M PB pH 7 was mixed with GO/PTZ. 104, of the mixture was cast on the glassy carbon electrode and GO was incubated in 4° C. for at least 12 hours to result in a dry film. Upon film formation, the electrode was equilibrated at R.T. for at least 30 minutes.

Prior to the amperometric measurements the modified electrode was electrochemically reduced by biasing the electrode potential to −0.85 V vs. Ag/AgCl for 200 seconds.

### Electrochemical Measurements

A conventional three electrode set-up was assembled with a graphite rod as counter electrode, modified glassy carbon disk electrode (3 mm in diameter; ALS, Tokyo, Japan) as working electrode and Ag/AgCl (NaCl 3M) electrode as a reference electrode (ALS, Tokyo, Japan). Cyclic voltammetry (CV) and amperometric detection was performed with a PalmSense potentiostat (Palm Instruments, Houten, The Netherlands) in 0.1M PB pH 7.

For the electrochemical study, rGO/PTZ/GDH/GCE was tested using different scan rates and the calibration curves of anodic peak potentials Eₚₐ as a function of ln ν and Iₚₐ as a function of ν were plotted.

### Fuel Cell Construction and Performance Characterization

The assembled electrodes served as the anode in a two compartment semi-biofuel cell (10 mL); consisting of 0.1M PB buffer (pH 7) in the anode and cathode chambers and glucose (100 mM) in the anode only. The cathode was potentiostatically controlled, using a three-electrode configuration: graphite rods as working electrode, and counter electrodes and Ag/AgCl as a reference electrode (ALS, Tokyo, Japan). The cathode was biased to a potential of +700 mV against Ag/AgCl. The voltage generated from the biofuel cell was measured by a hand held multimeter (DM-97, Sinometer, China). Various external resistances were applied between the anode and cathode by a resistance decade box (RBOX 408, Lutron Electronic Enterprise, Taipei, Taiwan). The generated voltage at each resistance was measured after reaching equilibrium. Measurements were carried out at ambient temperature.

### Phenothiazone Characterization

To verify successful oxidation of phenothiazine to phenothiazone the organic fraction was separated from the GO using preparative HPLC (Sapphire 600 instrument, ECOM) with a Luna C18 column, 10 μm (250×21.20 mm), at a flow rate of 20 mL/min. NMR analysis was performed using a Bruker Avance DPX400 or, alternatively, a Bruker Avance DMX500 spectrometer. LC-MS analyses were performed on an LCQ Fleet mass spectrometer (Thermo Scientific) with an ESI source. Spectra were collected in the positive ion mode and analyzed by Xcalibur software (Thermo Scientific). High resolution MS characterization was conducted using an Agilent 6520 high accuracy Quadrupole Time of Flight (QTOF) mass spectrometer. 5 μL injection volume was used. Acetonitrile was used as the mobile phase at 0.2 mL min⁻¹ flow rate. The analysis was conducted in both negative and positive mode using Agilent G3251A Dual ESI source. Nebuliser pressure was set to 40 psi, drying gas flow was 10 L min⁻¹, drying gas temperature 300° C., capillary voltage potential was 4000 V for the positive mode and 3000 V for negative mode. 0 V was set for nozzle voltage. The fragmentor voltage was set at 145 V, and skimmer voltage was 65 V. Scan range was 50-1000 m/z, and the other MS parameters remained at autotune conditions. The negative mode showed a signal of m/z 213.025 which corresponds to the formation of anion radical of phenothiazone in-situ in the electrospray in the negative mode. This point is not surprising in light of the reversible electrochemical behavior of this compound that is the subject of this paper. The results show the peak of phenothiazine in the positive mode. The insert presents the theoretical mass spectra for the molecule of phenothiazine with attached proton. The close similarity of the isotopic patterns (96%) and a very low mass error of isotopic masses between theoretical and experimental spectra (4.38 ppm) confirm the chemical formula of the compound (Cl₂H₇NSO).

¹³C NMR

¹³C NMR (MeOH, 400 MHzMHz): δ (ppm) 134.21 C2, 147.5 C3, 138.2 C5, 125.49 C6, 126.67 C7, 132.92 C8, 129.62 C9, 135.32 C10, 120.45 C11, 184.50 C12, 135.98 C13, 141.79 C14

¹H NMR

¹H NMR (400 MHz, METHANOL-d4) ppm 6.88 (d, J=2.20 Hz, 1H) 6.99 (dd, J=9.90, 2.20 Hz, 1H) 7.57-7.67 (m, 2H) 7.67-7.73 (m, 1H) 7.78 (d, J=9.90 Hz, 1H) 7.95-8.05 (m, 1H).

### Raman Spectroscopy

Measurements were performed at room temperature with a Renishaw inVia Raman microscope. The incident light was 514 nm with a power of 5.0 mW. Regular spectra were acquired with a 20× objective, and the line scan measurements were performed with a 5× objective. Spatial resolution for line scan was 5 μm.

X-ray photoelectron spectroscopy (XPS): XPS measurements were performed on a Kratos Axis Ultra x-ray photoelectron spectrometer (Manchester, UK). High-resolution spectra were acquired with a monochromated Al Kα (1486.6 eV) x-ray source with 0° takeoff angle. The pressure in the test chamber was maintained at 1.7×10⁻⁹ Torr during the acquisition process. Data analysis was performed with Vision processing data reduction software (Kratos Analytical Ltd) and CasaXPS (Casa Software Ltd).

### Example 1

### Cyclic Voltammograms of the Response of the GO/PTZ/GOx Biosensor to Different Glucose Concentrations

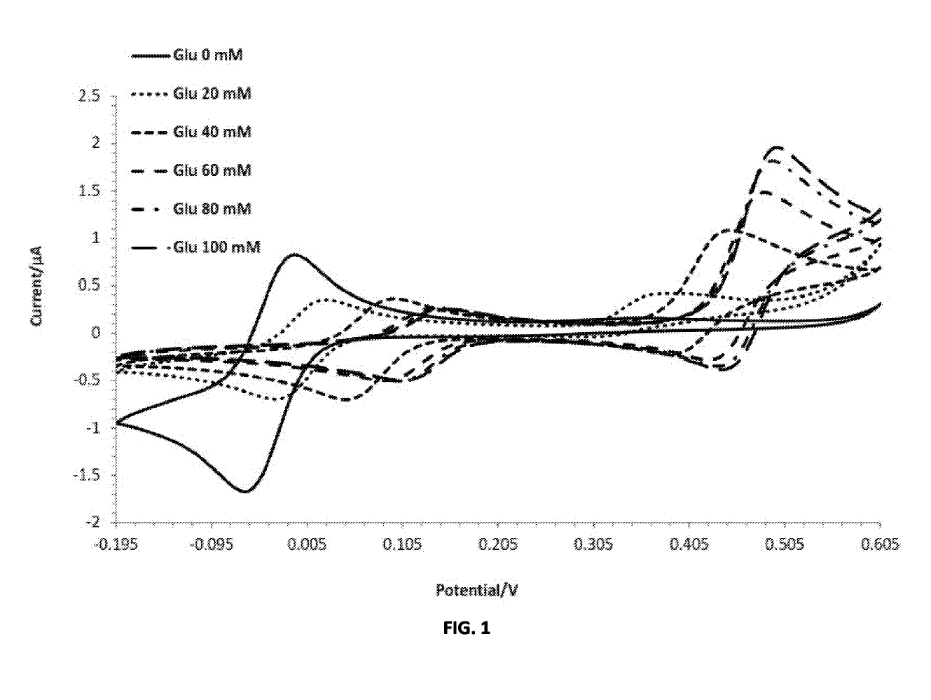

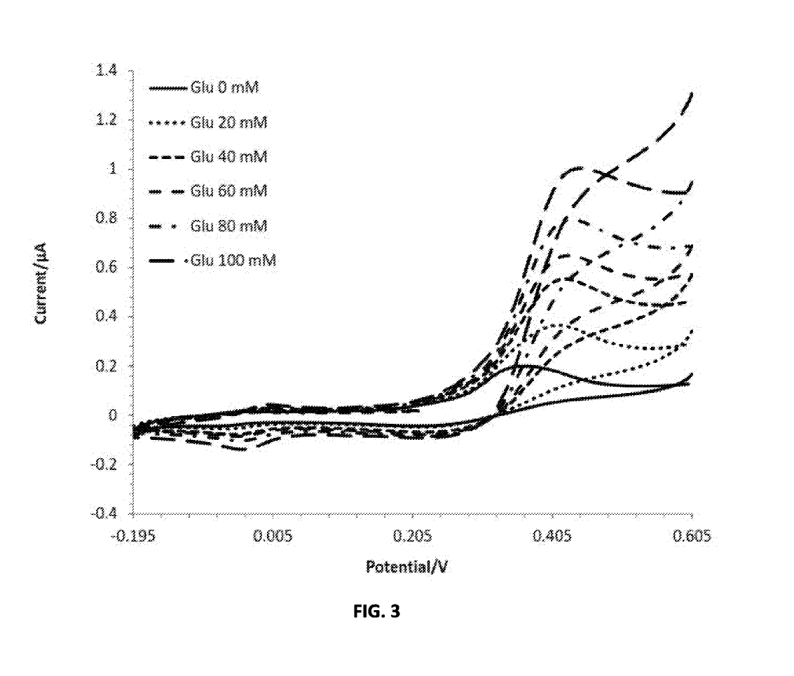

FIG. 1 shows cyclic voltammograms of the response of the GO/PTZ/GOx biosensor to different glucose concentrations. The left peek represent a modified form of the phenothiazine generated through the modified GO preparation and do not involve in the electrochemical response of the biosensor. The shift in potential in different glucose concentrations can be attributed to pH change because of the formation of gluconic acid through the catalytic activity of GOx. To verify this assumption analysis the system in different pH buffers was performed. FIG. 3 shows the positive shift upon increasing the acidity of the solution from pH 7 to pH 3. Notice that in the absence of glucose the positive peek seen in FIG. 1 is negligible.

### Example 2

### Cyclic Voltammograms of the Response of the PTZ/GOx Biosensor to Different Glucose Concentrations

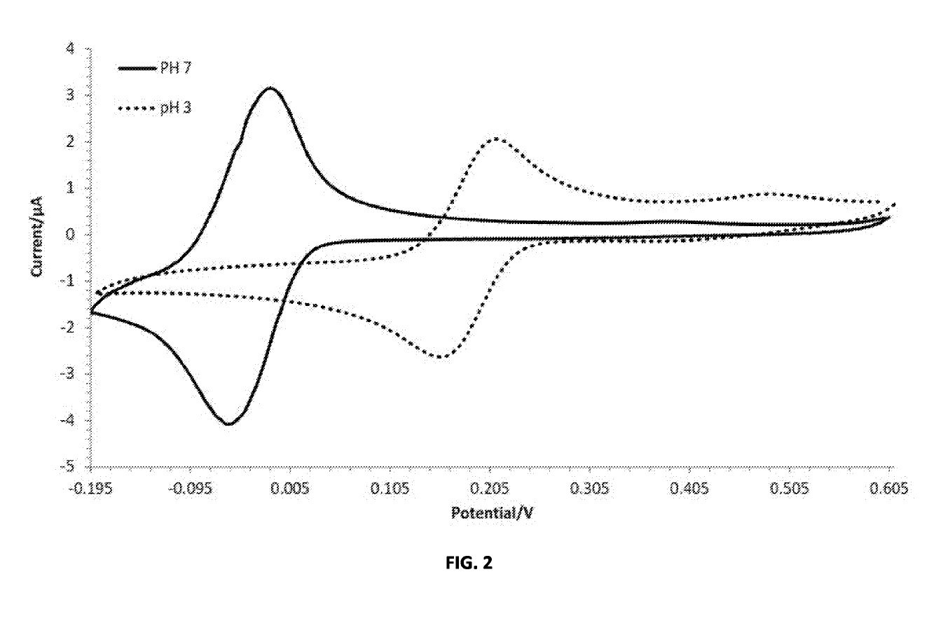

A similar biosensor omitting GO was constructed to examine the influence of GO on the system. As it can be seen in FIG. 2 there is a biocatalytic activity without GO but the current response is 50% lower.

This increase in response can be attributed to the high surface area and good conductivity of the GO after its reduction by the GOx activity.

### Example 3

The Effect of Engineered Yeast Expressing GOx on their Surface

An engineered yeast expressing GOx on their surface was prepared. A yeast surface display (YSD) system was utilized to express GOx on the surface of Saccharomyces cerevisiae (S. cerevisiae) yeast. FIG. 3 shows a biocatalytic activity of yeast expressing GOx in the presence of glucose. The low current may be attributed to low concentration of catalyst (GOx) due to low efficiency of induction in the YSD system. The YSD system can introduce many advantageous to the biosensor performance, among others: the efficient capability of the yeast to reduce the GO and thus to restore its advantageous electrical properties.

Because of the low diffusion coefficient of glucose in GO

$$( ∼ {10}^{- 12} \frac{{m}^{2}}{s} )$$

comparing to that in water

$$( ∼ {10}^{- 9} \frac{{m}^{2}}{s} ) ,$$

it was hypothesized that the diffusion can be a limiting factor in the system which exclude the glucose and enzymes from reaching the electrode and thus increasing the response time. To solve this problem, a new electrodes composed of GO film encapsulate engineered yeast were fabricated. In that way the diffusion of glucose occurs only in the thin film which is about 5 μm thick.

FIG. 3 demonstrates the efficient encapsulation of the yeast by the GO. The GO sheets are flexible enough to wrap together several yeast cells and connect clusters of microorganisms to nearby isolated cells or clusters.

In conclusion, a novel glucose biosensor made of GO/PTZ is introduced. The current results show a specific response to glucose but the biosensor can be adapted to sense other analytes by using different catalysts as GO/PTZ biocomposite can encapsulate different catalysts as well as glucose oxidase.

### Example 4

### Additional Biosensor Characterization

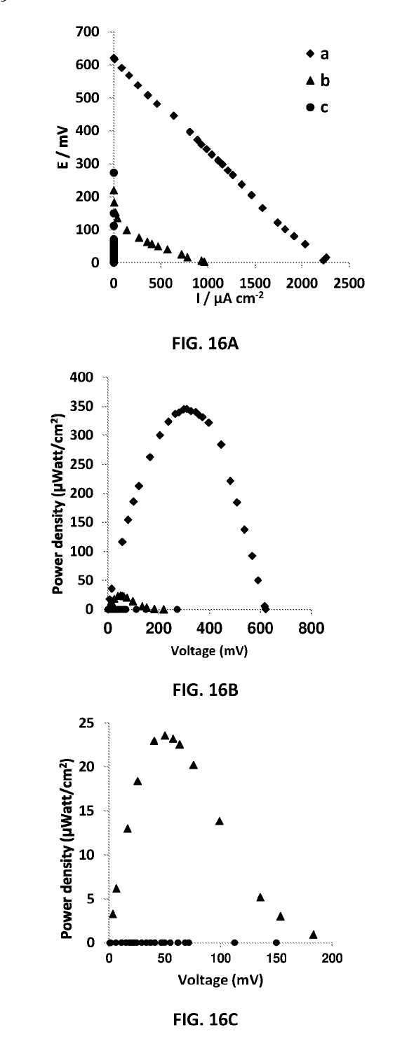

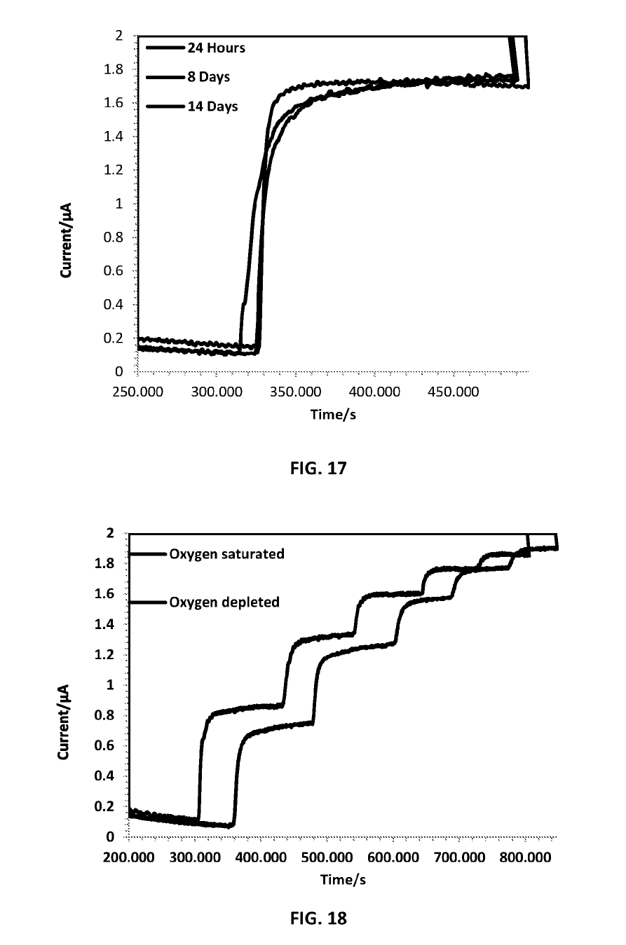

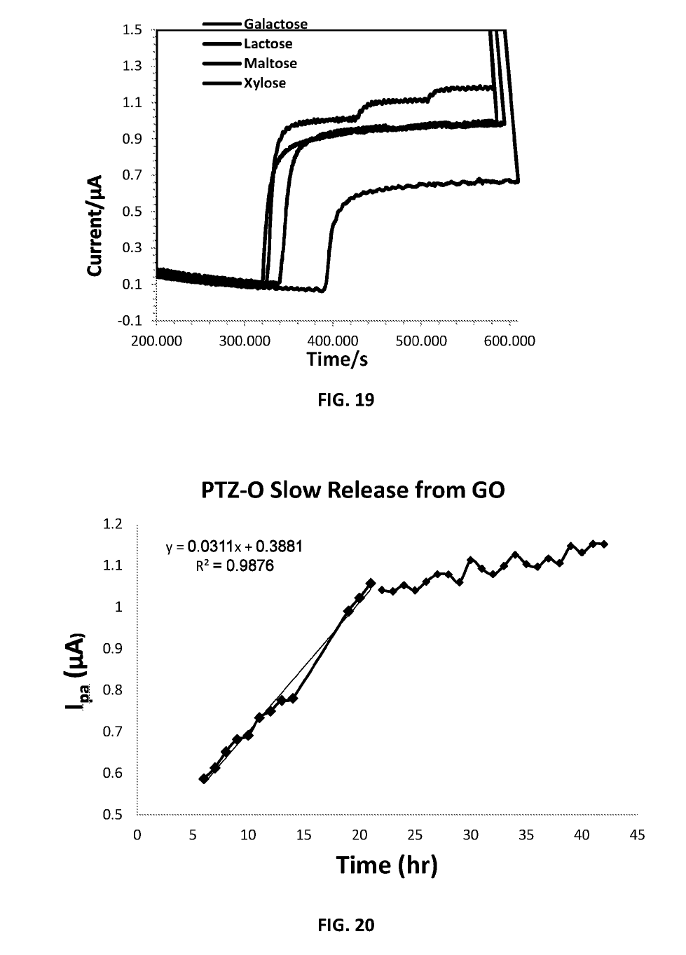

Reproducibility of the biosensor was tested by measuring the amperometric response to 3 mM glucose. Standard deviation for the response of 4 consecutive electrodes among 6 electrode tested was 0.08 indicating a good operational stability and thus a promising applicability for glucose sensing. Detection threshold was calculated to be 75 μM based on signal to noise ratio of 3 (S/N≧3). Long term stability of the electrode was tested and no change of the signal to 3 mM glucose was observed after 8 and 14 days storage at 4° C. (FIG. 18). Dehydrogenases often exhibit some degree of oxidase activity′. To verify that PTZ is not susceptible to oxidation by 02 the amperometric response of the rGO/PTZ/GDH modified electrode in the presence and in the absence of oxygen was tested (FIG. 19). The results suggest that the fabricated biosensor completely excluded 02 as an electron acceptor and thus is compatible for blood glucose monitoring. The sensitivity of rGO/PTZ/GDH/GCE to different sugars like galactose, lactose, xylose and maltose was also tested (FIG. 20). Only slight galactose interference was observed indicating a good selectivity of the biosensor to other sugars that may be present in human blood samples. Moreover, the selectivity to other interfering compounds like acetaminophen and ascorbic acid (AA) was tested. No sensitivity to acetaminophen was detected, however, the biosensor was sensitive to AA. To test the source of this activity towards AA, Bovine serum albumin (BSA) was used instead of GDH. BSA is a plasma protein rather than a redox enzyme. Hence, it was encapsulated in the rGO/PTZ matrix. An electrode response to AA was observed in the absence of GDH as well. AA can spontaneously oxidize on carbon electrodes however, GDH did not oxidize AA.

### Example 5

rGO/PTZ/GCE Characterization

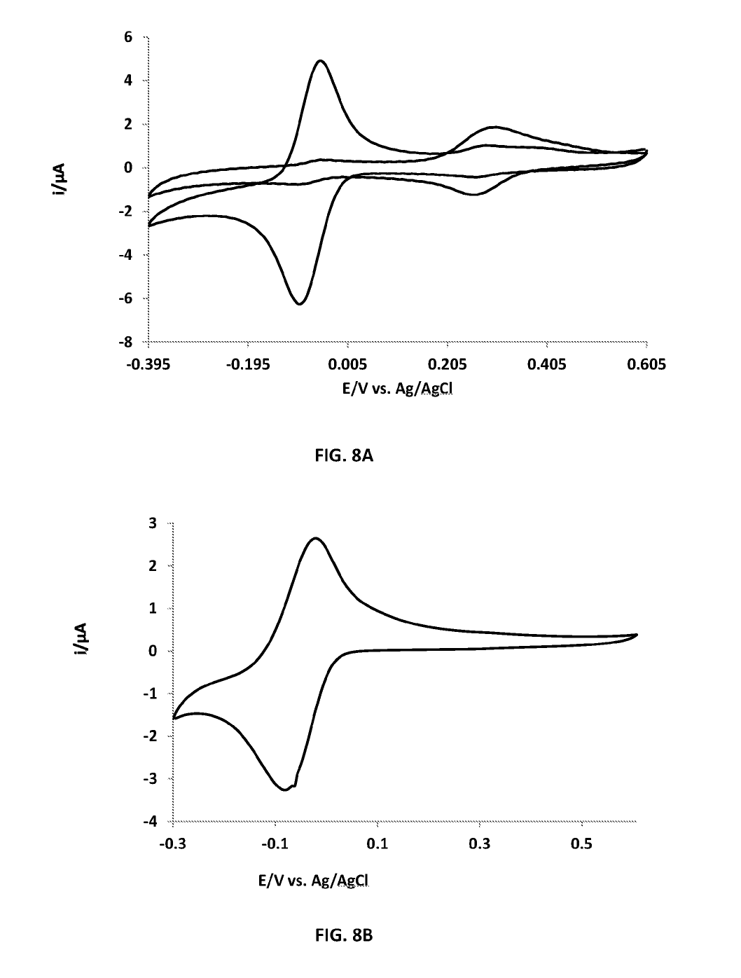

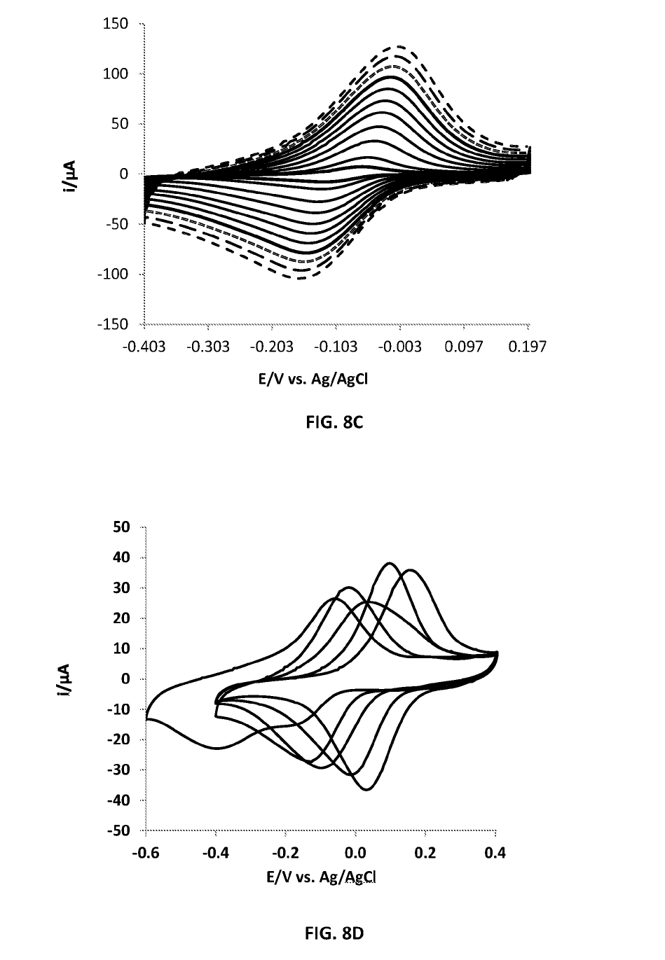

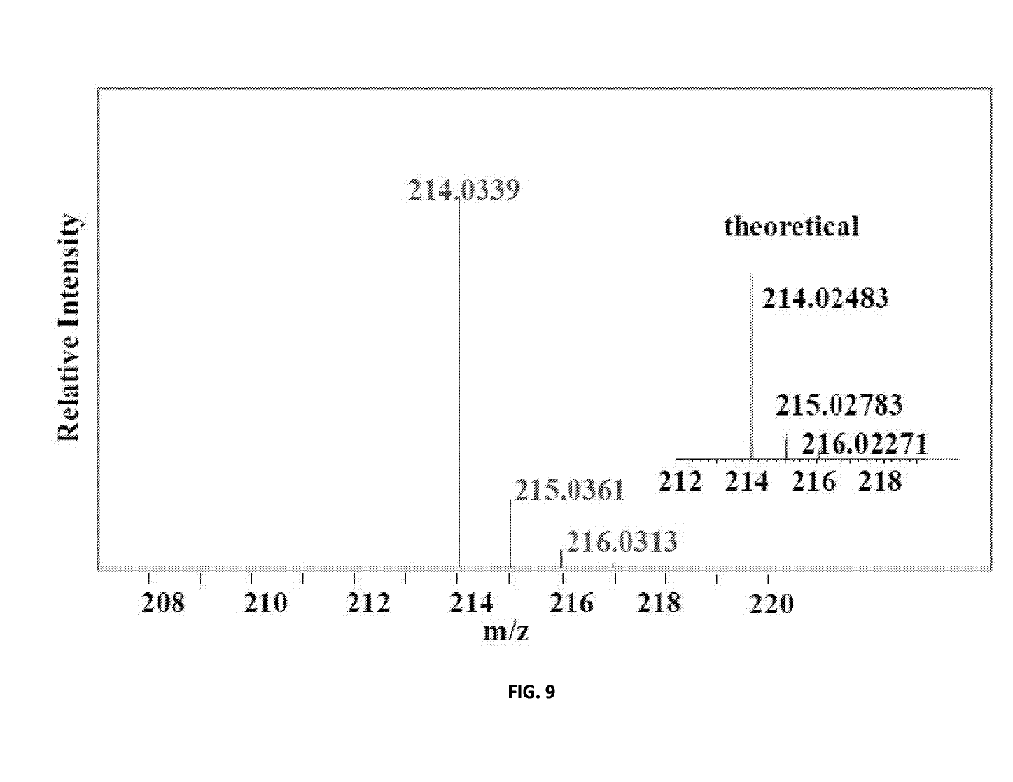

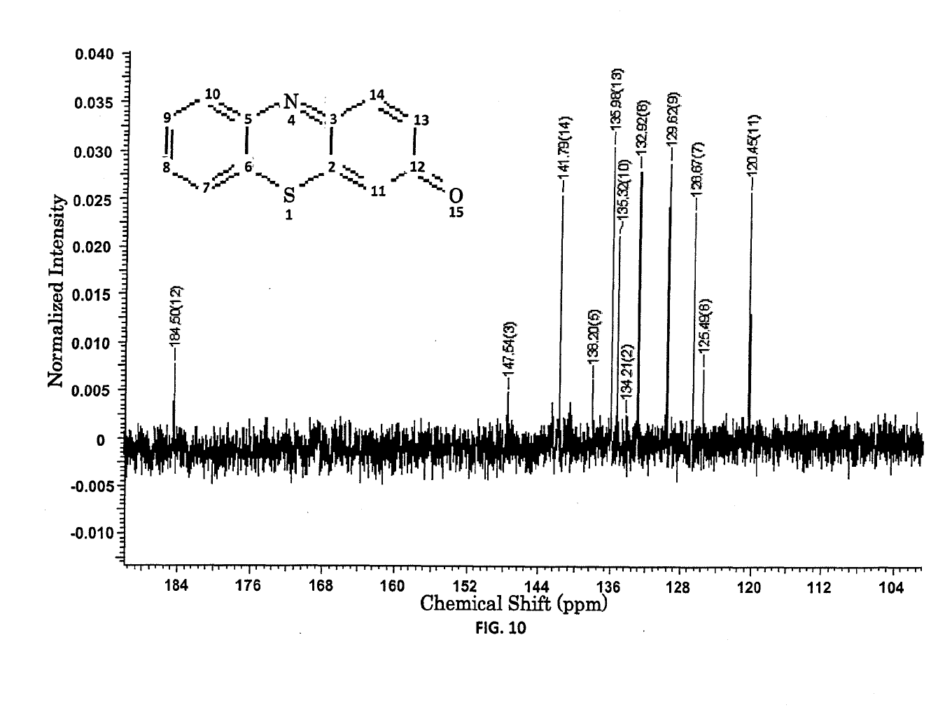

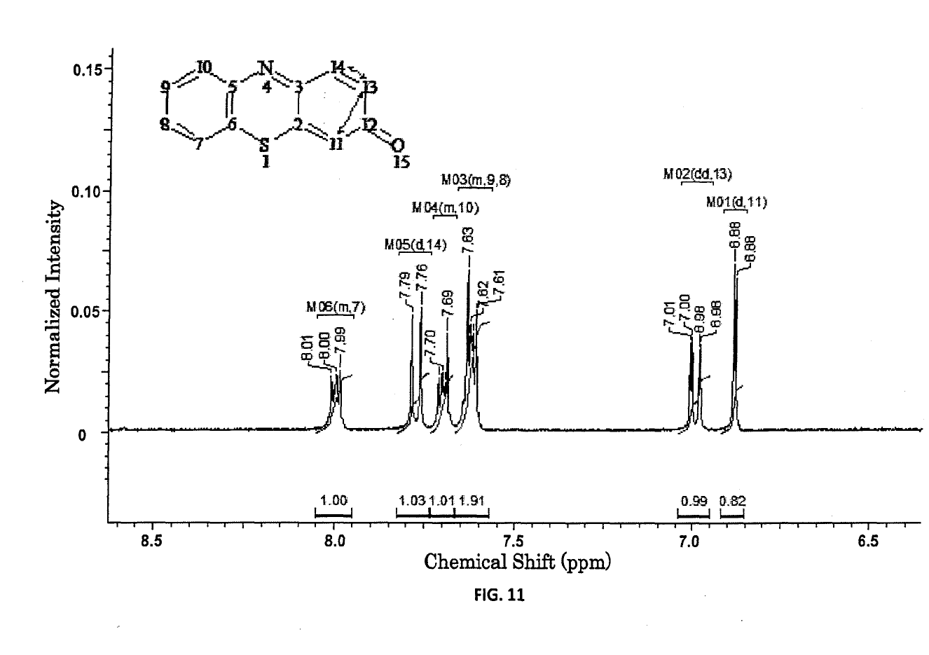

Electrochemical properties of the new composite material of rGO/PTZ/GCE have been studied by cyclic voltammetry (CV). FIG. 8(A) shows a CV of rGO modified with PTZ (rGO/PTZ) (a) and a solution with the soluble fraction of mostly insoluble PTZ (b) using a glassy carbon electrode (GCE) as the working electrode. The middle point potential (E1/2) of PTZ in an aqueous solution was calculated as the mean of the anodic peak (Epa) and cathodic peak (Epc) to be 280 mV vs. Ag/AgCl. Upon adsorption of PTZ on GO platelets, PTZ was oxidized to PTZ by GO, which in turn was partially reduced to rGO. In this process PTZ accumulated while PTZ has diminished (FIG. 8(A), curve a). For the purified PTZ extracted from the PTZ modified rGO matrix, a reversible electrochemical behavior was observed with a middle point potential of E1/2=−70 mV (FIG. 8(B)) and a peak-to-peak separation of ca. ΔE=60 mV which corresponds to a complete reversible behavior of a one electron redox process. In order to identify the oxidized PTZ, purified PTZ was characterized by H-NMR, C-NMR and LC-high resolution MS analysis (FIGS. 9-11 ESI). LC-MS fraction that was collected corresponded to a mass of 214.033 Da. Cyclic voltammetry was performed on the collected fraction and indeed confirmed that the molecule with a mass of 214.13 Da is the source of the newly generated redox peak. FIG. 12 shows the structure of PTZ (1) and PTZ as was determined by MS and NMR analyses. LC-MS analysis has also shown that another oxidized species exists in the GO upon graphene reduction and PTZ oxidation, a dimer shown in FIG. 12 (3), which is a combination of PTZ and PTZ-O. This combination was further verified by CV and it has shown a similar behavior as is seen for both PTZ and PTZ-O when put together in a solution with one peak at 280 mV and one peak at −70 mV. The dimer (3) however, is present in smaller quantities in the GO matrix and the dominant species is PTZ-O. In FIG. 8(B) a reversible voltamogram of pure PTZ-O can be observed. PTZ-O adsorbed in rGO shows reversible electrochemical behavior with a surface confined process behavior as indicated by the measurements shown in FIG. 8(C). CVs remain essentially unchanged on consecutive potential scan cycles indicating that rGO/PTZ-O modified electrode remains stable over time and polarization.

In order to determine the number of electrons transferred per molecule, the voltametric response of the modified electrode under pH values varying from 4 to 8 was tested (FIG. 8D). The formal potential is pH dependent with a negative shift with increasing pH (inset of FIG. 8D).

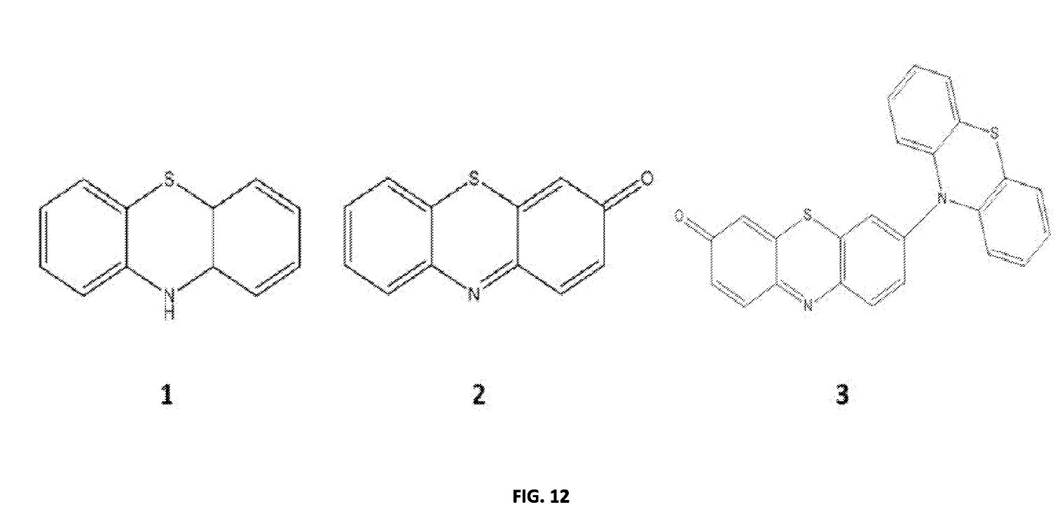

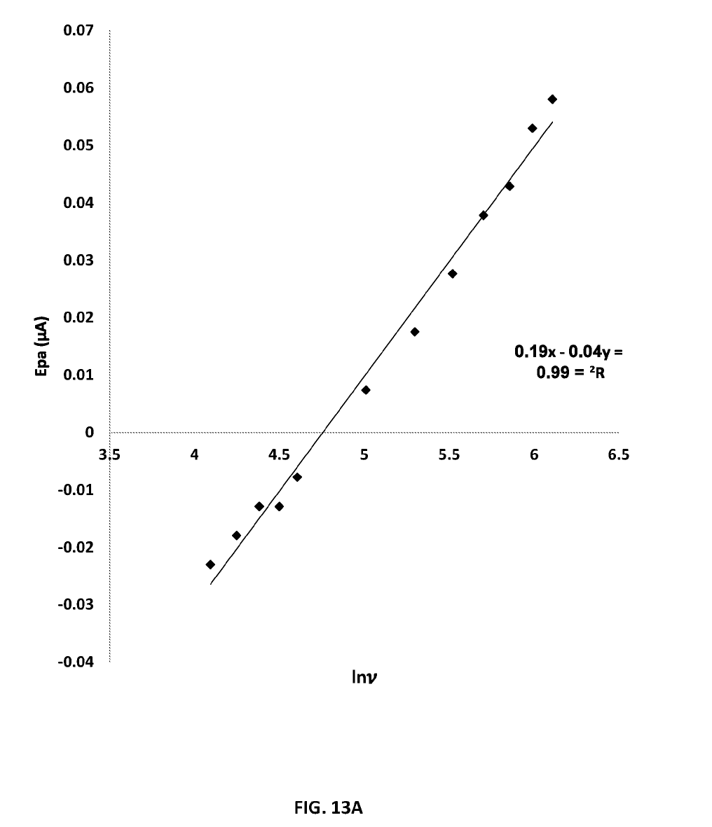

Results were plotted as E1/2 vs. pH, slope was calculated to be 52.9 mV/pH. This value is close to the Nernstian value of 59.2 mV/pH in the modified form of the Nernst equation which represents a 1 electron transfer process. The electron transfer coefficient (α) can be calculated according to Laviron's equation. In this case the anodic peak potential changed linearly vs. the natural logarithm of scan rate (v) in the range of 60 to 450 mV/s and α can be obtained from the slope of the curve (FIG. 13 (A)). An electron transfer coefficient of 0.37 was estimated for the reversible redox reaction indicating a good symmetry between the forward and reverse electron transfer step and an apparent electron transfer rate constant of kapp 0.535=s⁻¹. Based on the slope of the curve generated for Ip vs. the scan rate the average surface concentration (Γ) of the electro-active compound (PTZ-O) on the surface of rGO modified glassy carbon electrode was estimated (FIG. 13B). Using Laviron's theory the value of F was calculated to be 2×10−8 mol·cm−2. Comparing to other electroactive compounds immobilized on GCE it was concluded that a high number of PTZ molecules are successfully immobilized and available in this system. This high surface concentration can be attributed to the high surface area of GO nano-platelets and an efficient interaction between PTZ-O and rGO. In order to further verify the proposed model for GO reduction and PTZ oxidation was performed Raman spectroscopy measurements of the relative D and G bands of graphene. The G-band (around 1580 cm⁻¹) is common to all sp² carbon atoms, whereas the D-band is attributed to sp^{a} atoms and disordered structure of graphene. Indeed, the small changes in the D/G bands ratio cannot provide a clear indication for the reduction of the graphene oxide by PTZ (FIG. 14A).

Hence, verification the reduction by XPS was performed. FIG. 14(B) in the ESI section shows the XPS C1S binding energy spectra of GO samples before and after reduction by PTZ, the amount of oxygenated carbon atoms on the graphene has substantially decreased after exposure to PTZ.

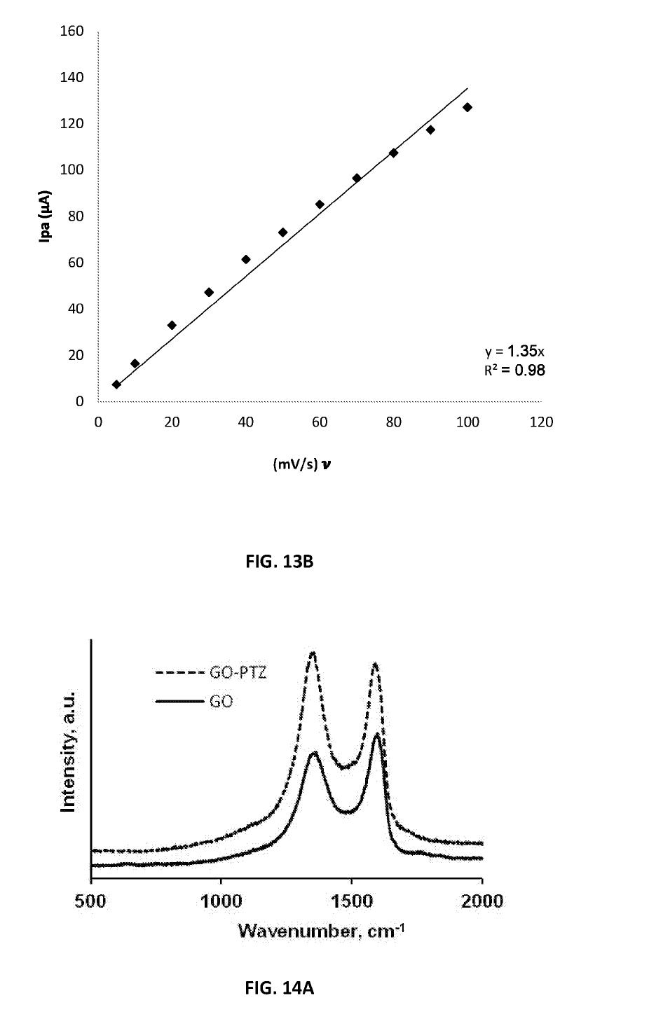

Enzymes can be immobilized efficiently by non-destructive entrapment in GO hydrogels. Based on the flexibility of GO hydrogel and robustness of PTZ-O as a reversible redox compound, a simple and facile method was developed to create a film-like bio-composite on the surface of glassy carbon electrode. In this way, a film modified electrode comprising of an enzyme, mediator and rGO was fabricated. To investigate the catalytic performance of rGO/PTZ bio-composite towards glucose oxidation, FAD-GDH was immobilized within the composite to serve as a biocatalyst. FAD-GDH from Bulkholderia cepacia was expressed in E. coli and was purified. FIG. 15 shows the catalytic activity of rGO/PTZ/GDH/GCE using cyclic voltammetry (CV). The enzymatic oxidation of glucose is visible as an anodic current with an onset potential of −0.35V and is related to the oxidation of FAD mediated by PTZ immobilized on rGO film. Next, the amperometric response of rGO/PTZ/GDH modified glassy carbon electrode was tested at increasing glucose concentrations. Electrochemical reduction (ER) was performed on the rGO film by applying a constant potential of −0.85 V for 200 seconds, resulting in an increase in capacitance and conductivity of the system while still keeping the enzyme activity intact. FIG. 15A shows the amperometric response of a biosensor for standard additions of 1, 2 and 5 mM glucose at an applied potential of 0.1 V. The duration and the potential of the electrochemical reduction were optimized to receive optimal signals and signal to noise ratios. As can be seen from FIG. 15A, upon electrochemical reduction the signal and response time are high and robust. The dynamic range of the ERrGO/PTZ/GDH modified GC electrode is between 0.5-40 mM and the linear range is 0.5-12 mM. Sensitivity was calculated from the slope of the linear part of the calibration curve and found to be ca. 34 mA M⁻¹cm⁻² which is among the highest reported values comparing to other glucose biosensors. Inset of FIG. 15A shows a calibration curve of the system using different glucose concentrations.

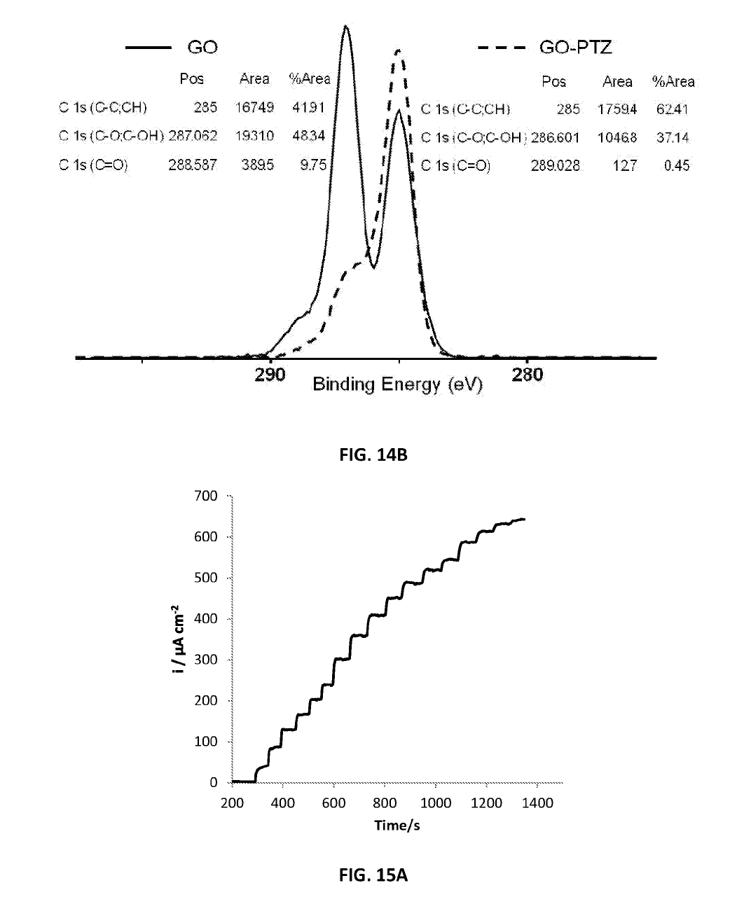

Next, the ERrGO/PTZ/GDH modified GC electrode performance was tested as an anode in a biofuel cell. To eliminate limitations from the cathode, cathode was controlled by a potentiostat and was biased continuously to 400 mV. FIG. 16 shows the polarization curve and power outputs of a biofuel cell constructed from ERrGO/PTZ/GDH modified GCE.

From FIG. 16B it can be seen that power output generated by GDH encapsulated in ERrGO/PTZ matrix is as high as 345 μW/cm². This impressive performance can be attributed to the robustness of GDH, GO and PTZ, efficient electron transfer between GDH and rGO/PTZ and increase in conductivity of the rGO by the further electrochemical reduction and thus increase in electron transfer rates and efficiency. As control experiments glucose oxidase (GOx) was entrapped instead of GDH in the rGO/PTZ matrix or used no enzyme at all. ERrGO/PTZ/GOx bioanode produced a power output of 23.6 μW/cm², a decrease of more than 90% in power compared to the 345 μW/cm² produced by ERrGO/PTZ/GDH bioanode. This is an indication of the efficient utilization of PTZ as a mediator by GDH. The fill factor (f) of the biofuel cell using ERrGO/PTZ/GDH as a bioanode was calculated to be ca. 25%. The low fill factor indicates a significant deviation from the optimal rectangular-shaped polarization curve. This observed deviation can be explained by the mass transport loss caused by leakage of active compounds from the electrode during a long term operation.

Thus a novel bio-composite made of rGO and PTZ produced using a facile procedure, which mediates an efficient, and robust electron transfer between the FAD co-factor of GDH and an electrode is introduced herein. The high sensitivity, stability and selectivity render the system promising for glucose biosensing. The ability of rGO to encapsulate enzymes and form a film contributes to its appeal in future biosensing applications. Moreover, the low operating potential of the system as well as good communication of GDH with the mediator enables the system to serve as an efficient bio-anode for the electro-catalytic oxidation of glucose in a biofuel cell. The robustness of modified graphene oxide and the flexibility of the hydrogel to entrap bio-molecules demonstrate its great potential in bio-electronic systems.

### Example 6

### PTZ-O—Phenothiazone Extended Release Formulation

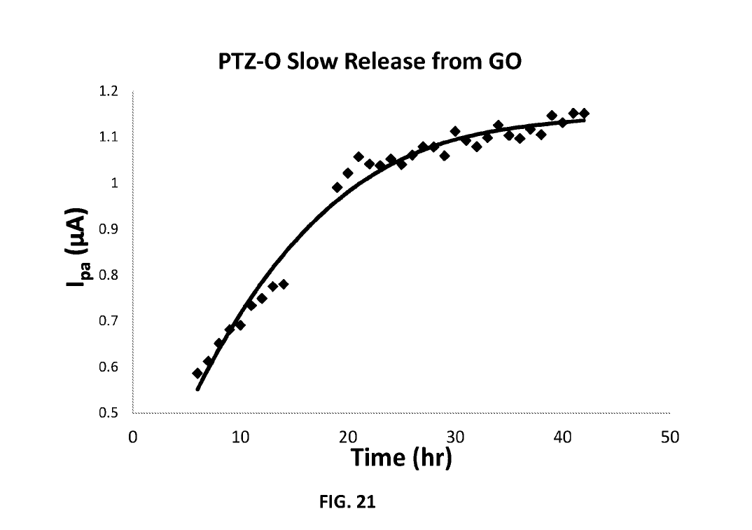

In this example, each point in the graph (FIGS. 20 and 21) represent the oxidation peak current of the cyclic voltammogram taken every one hour using glassy carbon, graphite rod and Ag/AgCl (3M KCl) as working, auxiliary and reference electrodes respectively.

Specifically, a combined preparation of GO/PTZ-O was inserted into cellulose membrane (dialysis) and placed in a stirred PB (100 mM) solution pH 7. The three electrodes were placed in the PB solution and measurements were taken every one hour.

FIGS. 20 and 21 show that combining PTZ-O and a GO matrix results in an extended/slow PTZ-O release composition. As indicated from the saturation curve, when ca. 10 mM PTZ adsorbed in GO matrix, it slowly release and reach saturation after ca. 21 hours. In the process of PTZ adsorption by GO, oxidation-reduction process occurred in which GO is partially reduced to rGO and most of the PTZ adsorbed was oxidized to PTZ-O.

As opposed to PTZ, PTZ-O is highly soluble in water solutions, thus releases from the matrix when introduced to a polar solvent. In this way, a slow/extended release system is introduced with a saturation curve.

As PTZ and its derivatives are known as drugs for, inter-alia, psychotic syndromes, skin diseases and multiple other ailments, a system in which PTZ and derivatives and PTZ-O and derivatives can be slowly released is of great importance for the pharmaceutical industry. Likewise, this system/composition provided an additional, utmost, valuable feature which includes the substantial enrichment of PTZ-O (as the initial reduced form PTZ is converted to the medically active PTZ-O in the presence of GO). Moreover, the biocompatibility of graphene oxide enables the system to be introduced in-vivo as a drug and even transplanted. Once introduced in-vivo, PTZ-O will be released over time (see FIGS. 20 and 21) in the human body.

1. A composition comprising graphene oxide (GO) and PTZ.

2. The composition of claim 1, wherein said PTZ is: phenothiazine, phenothiazone, or a combination thereof.

3. The composition of claim 1, further comprising a redox enzyme.

4. The composition of claim 3, wherein said redox enzyme is entrapped within a composite comprising said graphene oxide and said PTZ.

5. The composition of claim 1, wherein said graphene oxide is reduced graphene oxide.

6. The composition of claim 1, wherein said PTZ comprises: less than 10% reduced form of PTZ (phenothiazine), at least 90% oxidized PTZ (phenothiazone) or both.

7. The composition of claim 1, further comprising flavin adenine dinucleotide (FAD).

8. The composition of claim 1, wherein said composition is a controlled release or extended release composition.

9. The composition of claim 1, further comprising a sample comprising a bodily fluid.

10. The composition of claim 1, further comprising gluconic acid, glucose or both.

11. An electrode comprising the composition of claim 1.

12. (canceled)

13. (canceled)

14. (canceled)

15. (canceled)

16. A method for extending the release of PTZ in a physiological environment, comprising the step of combining said PTZ and GO.

17. (canceled)

18. (canceled)

19. (canceled)

20. A method for treating a subject afflicted with: a psychiatric disease, inhibiting leukotriene biosynthesis, treating pain, reducing inflammation, treating allergy, treating a pulmonary disease, treating asthma, treating a cardiovascular disorder, or any combination thereof, comprising administering to said subject the composition of claim 1, thereby treating a subject afflicted with: a psychiatric disease, inhibiting leukotriene biosynthesis, treating pain, reducing inflammation, treating allergy, treating a pulmonary disease, treating asthma, treating a cardiovascular disorder, or any combination thereof.

21. (canceled)

22. (canceled)

23. A method for converting phenothiazine to phenothiazone comprising the step of contacting graphene oxide (GO) and phenothiazine, thereby converting phenothiazine to phenothiazone.

24. (canceled)

25. (canceled)

26. (canceled)

27. The method of claim 23, wherein said mixture comprises phenothiazone.

28. The method of claim 23, resulting in a mixture having a ratio (weight/weight) of phenothiazone to phenothiazine of at least 9:1.

29. A method for detecting, quantifying or both, a carbohydrate in a sample, comprising the steps of:
(a) providing the composition of claim 1, the composition further comprising a redox carbohydrate enzyme, said carbohydrate is a substrate of said redox carbohydrate enzyme;
(b) contacting the composition with said sample; and
(c) subjecting the mixture obtained in step (a) to an electric potential;
thereby detecting, quantifying or both, a carbohydrate.

30. A kit comprising the composition of claim 1 and means for connecting said composition to an electrical source.
